SEQUENCE LISTING (1 sequence)

SEQ ID NO: 1 - protein, 34 aa, synthetic construct
  SITE 2: note = Aib
  MOD_RES 20: note = Lys at position 20 is chemically modified by conjugation of the epsilon-amino group of the Lys side chain with ([2-(2-aminoethoxy)-ethoxy]-acetyl)2-(gamma-Glu )-CO-(CH2)18CO2H
  MOD_RES 34: note = C-terminal amino acid is amidated
HXQGTFTSDYSKYLDEKKAKEFVEWLLEGGPSSG

(19) United States
(12) Patent Application Publication
(10) Pub. No.: US 2026/0248879 A1
(43) Pub. Date: Aug. 27, 2026

(54) METHODS OF USING A GCG/GLP1 CO-AGONIST FOR THERAPY

(71) Applicant: ELI LILLY AND COMPANY, Indianapolis, IN (US)

(72) Inventors: Melissa Kay THOMAS, Indianapolis, IN (US); Wei NI, Indianapolis, IN (US); Deborah Ann ROBINS, Fortville, IN (US); Lai San THAM, Singapore (SG)

(21) Appl. No.: 18/870,122

(22) PCT Filed: May 31, 2023
(86) PCT No.: PCT/US2023/067652

§ 371 Date: Nov. 27, 2024

Related U.S. Application Data
(60) Provisional application No. 63/347,834, filed on Jun. 1, 2022.

(51) Int. Cl. A61K 38/16; A61K 9/00; A61P 3/04; A61P 3/06; A61P 3/10

(52) U.S. Cl. CPC A61K 38/16; A61K 9/0019; A61P 3/04; A61P 3/06; A61P 3/10

(57) ABSTRACT

Doses and dosing regimens for incretin analogs are disclosed comprising determining and administering doses of long-acting incretin analogs suitable for once-weekly dosing, such as a glucagon-like peptide-1 (GLP-1) and glucagon (Gcg) co-agonist.

### REFERENCE TO SEQUENCE LISTING

The disclosure is being filed along with a Sequence Listing in ST.26 XML format. The Sequence Listing is provided as a file titled “30324_WO_SeqListing” created May 18, 2023 and 3.1 kilobytes (kb) in size. The Sequence Listing information in the ST.26 XML format is incorporated herein by reference in its entirety.

### TECHNICAL FIELD

The present disclosure relates to methods of using, medical uses of, and pharmaceutical compositions having an incretin analog, or a pharmaceutically acceptable salt thereof, with activity at each of a glucagon (Gcg) receptor and a glucagon-like peptide-1 (GLP1) receptor, especially to methods of using and medical uses of certain doses of an incretin analog and compositions containing certain doses of an incretin analog that can be used for treating conditions, diseases and disorders including diabetes mellitus (especially type 2 diabetes mellitus (T2DM)) and/or symptoms thereof, obesity, hypertension, cardiovascular disease, in particular, MACE (which includes death, myocardial infarction; stroke, hospitalization because of heart failure; and revascularization, including percutaneous coronary intervention, and coronary artery bypass graft) and atherosclerosis, chronic kidney disease, diabetic kidney disease, osteoarthritis, polycystic ovary syndrome, dyslipidemia, non-alcoholic steatohepatitis (NASH), non-alcoholic fatty acid liver disease (NAFLD), obstructive sleep apnea and others.

### BACKGROUND

Over the past several decades, the prevalence of diabetes mellitus continues to rise, which is a chronic disorder characterized by hyperglycemia resulting from defects in insulin secretion, insulin action, or both. T2DM is the most common form of diabetes, accounting for about 90% of all diabetes. In T2DM, the combined effects of impaired insulin secretion and insulin resistance are associated with elevated blood glucose levels.

Uncontrolled diabetes can lead to one or more conditions that impact morbidity and mortality of such individuals. One of the main risk factors for T2DM is obesity, and many individuals with T2DM (~90%) are overweight or obese. It is documented that a decrease in body adiposity will lead to improvement in obesity-associated comorbidities. The current standard of care for T2DM, obesity and obesity related metabolic disorders include diet and exercise, as well as treatment with oral medications and injectable glucose-lowering drugs including incretin-based therapies, such as GLP-1 receptor agonists. Despite the success of the GLP-1 receptor agonists, significant numbers of individuals receiving approved therapies are not reaching their weight loss and/or glycemic control goals (see, e.g., Casagrande et al. (2013) Diabetes Care 36:2271-2279) and there remains a ‘gap’ between the efficacy of GLP-1 analogues and that of bariatric surgery. To plug this gap, researchers have pursued the ‘co-agonist’ strategy by combining GLP-1 with related hormones from the proglucagon family and related peptides, including GIP and Gcg itself (see Sanchez-Garrido M A et al., Diabetologia (2017) 60(10):1851-61).

There are multiple GLP-1/Gcg co-agonists in clinical development, including cotadutide, efinopegdutide, mazdutide (which is described in International Patent Application Publication No. WO 2016/209707 A1), SAR425899, BI 456906, JNJ-54729518, HM15211, NNC9204-1706, Alt-801 (pemvidutide) and G3215 (see Table 1 of Hope D C D et al., Frontiers in Endocrinology (Lausanne), 2021 Sep. 8; 12:735019). Certain of these GLP-1/Gcg co-agonists have been shown to have promising weight loss, glycemic effects or both in these early phase clinical results. However, some reports suggest that the weight loss and/or glucose lowering effects of these co-agonists may plateau such that higher doses do not result in improved efficacy, thus limiting achievable weight loss and/or glycemic improvement; moreover, the tolerability may be limited at high doses by adverse gastrointestinal events. See e.g. Ji et al., The Lancet, EClinicalMedicine 39 (2021) 101088; Nahra et al., Diabetes Care. 2021; 44:1433-42; Alba et al., Clinical Obesity, 2021 April; 11(2):e12432); Ambery et al., British Journal of Clinical Pharmacology, 2018 October; 84(10):2325-2335.

Therefore, there is a need for more efficacious doses and dosing regimens for GLP-1/GCG co-agonists that are able to deliver high weight loss and/or provide improved glycemic control in an individual in need of such treatment, while also preserving an overall acceptable benefit/risk profile to the individual.

### BRIEF SUMMARY

The present invention reports doses and dosing regimens for a GLP-1/Gcg co-agonist that result in unexpectedly improved weight loss, improved body composition, robust glucose control, lipid lowering and/or insulin sensitization sparing beta-cell insulin secretory workload while at the same time exhibiting only mild or moderate adverse events.

In one aspect, the present invention provides a method for providing chronic weight management, improving glycemic control, and/or reducing LDL cholesterol or triglycerides, in a patient in need thereof, wherein said method comprises:
(1) administering a once weekly dose of a compound of the following formula:

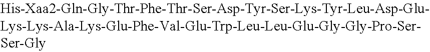

wherein Xaa2 is Aib;
Lys at position 20 is chemically modified by conjugation of the epsilon-amino group of the Lys side chain with ([2-(2-aminoethoxy)-ethoxy]-acetyl)₂-(γ-Glu)-CO—(CH₂)₁₈CO₂H; and
the C-terminal amino acid is amidated (SEQ ID NO:1),
or a pharmaceutically acceptable salt thereof,
to the patient in need thereof for at least about two weeks; and
(2) thereafter increasing the dose to greater than about 6 mg to about 16 mg and administering the increased dose to said patient once weekly for at least about two weeks.

In some embodiments, provided herein is a method for preventing and/or treating a condition selected from type 2 diabetes, hyperglycemia, non-insulin dependent diabetes, obesity; dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, reducing body weight, reducing food intake, and/or inducing satiety in a patient, wherein said method comprises:
(1) administering a once weekly dose of a compound of SEQ ID NO:1, or a pharmaceutically acceptable salt thereof, to a patient in need thereof for at least about two weeks; and
(2) thereafter increasing the dose to greater than about 6 mg to about 16 mg and administering the increased dose to said patient once weekly for at least about two weeks.

In another aspect of the present disclosure, there is provided a compound of SEQ ID NO: 1, or a pharmaceutically acceptable salt thereof, for use in providing chronic weight management, improving glycemic control and/or reducing LDL cholesterol or triglycerides, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In some embodiments, there is provided a compound of SEQ ID NO:1, or a pharmaceutically acceptable salt thereof, for use in preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, for use in reducing body weight, for use in reducing food intake and/or for use in inducing satiety in a patient, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In a further aspect of the present disclosure, there is provided the use of a compound of SEQ ID NO:1, or a pharmaceutically acceptable salt thereof, in the manufacture of a medicament for providing chronic weight management, improving glycemic control, and/or reducing LDL cholesterol or triglycerides, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In some embodiments, there is provided the use of a compound of SEQ ID NO:1, or a pharmaceutically acceptable salt thereof, in the manufacture of a medicament for preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, reducing body weight, reducing food intake and/or inducing satiety in a patient, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In a further aspect of the present disclosure, the once weekly increased dose is from about 6.25 mg to about 16.0 mg, from about 6.5 mg to about 16.0 mg, from about 8.0 mg to about 16.0 mg, from about 10 mg to about 16.0 mg, from about 11.0 mg to about 16 mg, from about 12.0 mg to about 16.0 mg, from about 13.0 mg to about 16.0 mg or from about 14.0 mg to about 16.0.

In a further aspect of the present disclosure, the once weekly increased dose is about 6.25 mg, about 6.5 mg, about 7.0 mg, about 8.0 mg, about 9.0 mg, about 10.0 mg, about 11.0 mg, about 12.0 mg, about 13.0 mg, about 14.0 mg, about 15.0 mg or about 16.0 mg. In some embodiments, the once weekly increased dose is about 6.5 mg. In some embodiments, the once weekly increased dose is about 7.0 mg. In some embodiments, the once weekly increased dose is about 8.0 mg. In some embodiments, the once weekly increased dose is about 9.0 mg. In some embodiments, the once weekly increased dose is about 10.0 mg. In some embodiments, the once weekly increased dose is about 11.0 mg. In some embodiments, the once weekly increased dose is about 12.0 mg. In some embodiments, the once weekly increased dose is about 13.0 mg. In some embodiments, the once weekly increased dose is about 14.0 mg. In some embodiments, the once weekly increased dose is about 15.0 mg. In some embodiments, the once weekly increased dose is about 16.0 mg.

In a preferred aspect of the present disclosure, the once weekly increased dose is from about 8.0 mg to about 16.0 mg. In a still further aspect of the present disclosure, the once weekly increased dose is about 8.0 mg, about 10.0 mg, about 11.0 mg, about 12.0 mg, about 13.0 mg, about 14.0 mg, about 15.0 mg or about 16.0 mg.

In another preferred aspect of the present disclosure, the once weekly increased dose is from about 10.0 mg to about 16.0 mg. In a still further aspect of the present disclosure, the once weekly increased dose is about 10.0 mg, about 11.0 mg, about 12.0 mg, about 13.0 mg, about 14.0 mg, about 15.0 mg or about 16.0 mg.

In one aspect of the present disclosure, the once weekly dose in step (1) is about 1.0 mg to about 6.0 mg. In a still further aspect of the present disclosure, the about weekly dose is about 1.0 mg about 1.5 mg, about 2.0 mg, about 2.5 mg, about 3.0 mg, about 3.5 mg, about 4.0 mg, about 5.0 mg, about 5.5 mg or about 6.0 mg.

In a still further aspect of the present disclosure, multiple different once weekly doses are administered to the patient in steps (1) and (2).

In one aspect of the present disclosure, the patient is administered a first or initial once weekly dose of about 1.0 mg to about 6.0 mg. Thus, in some embodiments, a first once weekly dose is about 1.0 mg to about 6.0 mg. In a further aspect of the present disclosure, the first once weekly dose is about 1.0 mg, about 1.5 mg, about 2.0 mg, about 2.5 mg, about 3.0 mg, about 3.5 mg, about 4.0 mg, about 4.5 mg, about 5.0 mg, about 5.5 mg or about 6.0 mg. In a preferred aspect of the present disclosure, the first once weekly dose is about 1.0 mg, about 1.5 mg, about 2.0 mg or about 3.0 mg. In some embodiments, the first once weekly dose is about 1.0 mg. In some embodiments, the first once weekly dose is about 1.5 mg. In some embodiments, the first once weekly dose is about 2.0 mg. In some embodiments, the first once weekly dose is about 3.0 mg.

In one aspect of the present disclosure, the first dose and each subsequent increased dose are increased in increments of about 1.5 mg, about 2.0 mg, about 2.5 mg, about 3.0 mg, about 3.5 mg, about 4.0 mg, about 4.5 mg, about 5.0 mg, about 5.5 mg, about 6.0 mg or any combination thereof, and wherein each dose is administered for at least about two weeks. Thus, the first dose is selected from about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, and about 6.5 mg, and each subsequent dose is increased over the previously administered dose in an increment of about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg or any combination thereof. In a preferred aspect, the first dose is selected from about 1.5 mg, about 2.0 mg, and about 3.0 mg and each subsequent dose is increased over the previously administered dose in an increment of about 1.5 mg, about 2.0 mg, about 3.0 mg, about 4.0 mg or any combination thereof.

In another aspect of the present disclosure, the dose may be lowered after administering the maximal or highest dose for that patient. For instance, once weekly doses of 2.0 mg, 4.0 mg, 6.0 mg, 8.0 mg and 10.0 mg of the compound of SEQ ID NO:1, or a pharmaceutically acceptable salt thereof, are administered to the patient, each for three weeks. After three weeks at the maximal dose of 10 mg, the dose is reduced to 8 mg.

In another aspect of the present disclosure, the method or use comprises administering each once weekly dose for at least about four weeks. In a still further aspect of the present disclosure, the method or use comprises administering each once weekly dose for at least about six weeks. In a still further aspect of the present disclosure, the method or use comprises administering each once weekly dose for at least about eight weeks.

In one preferred aspect, the method or use comprises administering each dose once weekly for at least about two weeks and administering the maximal dose once weekly for at least about four weeks. In another preferred aspect, the method or use comprises administering each dose once weekly for at least about 4 weeks and administering the maximal dose once weekly for at least about 4 weeks. In another preferred aspect, the method or use comprises administering each dose once weekly for at least about 4 weeks and administering the maximal dose once weekly for at least about 8 weeks.

In another aspect, the administration of the compound of SEQ ID NO:1, or a pharmaceutically acceptable salt thereof, results in a decrease in the patient's body weight. In some embodiments, the administration of the compound of SEQ ID NO:1, or a pharmaceutically acceptable salt thereof, results in at least about 10% decrease in the patient's body weight. In some embodiments, the administration of the compound of SEQ ID NO: 1, or a pharmaceutically acceptable salt thereof, results in at least about 15% decrease in the patient's body weight. In some embodiments, the administration of the compound of SEQ ID NO: 1, or a pharmaceutically acceptable salt thereof, results in at least about 20% decrease in the patient's body weight.

In another aspect, the administration of the compound of SEQ ID NO:1, or a pharmaceutically acceptable salt thereof, results in a decrease in the patient's HbA1c. In another aspect, the administration of the compound of SEQ ID NO:1, or a pharmaceutically acceptable salt thereof, results in a decrease in the patient's LDL cholesterol and/or triglycerides decreases. In another aspect, the administration of the compound of SEQ ID NO:1, or a pharmaceutically acceptable salt thereof, does not result in unacceptable tolerability.

In one aspect, the compound of SEQ ID NO:1, or a pharmaceutically acceptable salt thereof, is administered subcutaneously.

In one aspect, the patient is overweight. In another aspect, the patient is obese. In another aspect, the patient has type 2 diabetes.

### FIGURES

FIG. 1 illustrates the disposition of the participants in the clinical study of Compound 1 described in Example 1.

FIG. 2 illustrates the mean body weight changes from baseline in the placebo, Cohort 1 and Cohort 2 treatment groups in the clinical study of Compound 1 described in Example 1.

FIG. 3 illustrates the mean waist circumference changes from baseline in the placebo, Cohort 1 and Cohort 2 treatment groups in the clinical study of Compound 1 described in Example 1.

FIG. 4 illustrates the mean fasting glucose for placebo, Cohort 1 and Cohort 2 treatment groups in the clinical study of Compound 1 described in Example 1.

FIG. 5 illustrates the mean fasting insulin for placebo, Cohort 1 and Cohort 2 treatment groups in the clinical study of Compound 1 described in Example 1.

FIG. 6 illustrates the mean fasting C-peptide for placebo, Cohort 1 and Cohort 2 treatment groups in the clinical study of Compound 1 described in Example 1.

FIG. 7a illustrates the mean fasting cholesterol for placebo, Cohort 1 and Cohort 2 treatment groups in the clinical study of Compound 1 described in Example 1.

FIG. 7b illustrates the mean cholesterol profiles for placebo, Cohort 1 and Cohort 2 treatment groups after sMMTT performed as part of the clinical study of Compound 1 described in Example 1.

FIG. 7c illustrates the percent change from baseline (Day 1) for fasting HDL for the placebo, Cohort 1 and Cohort 2 treatment groups in the clinical study of Compound 1 described in Example 1.

FIG. 7d illustrates the percent change from baseline (Day 1) for fasting LDL for the placebo, Cohort 1 and Cohort 2 treatment groups in the clinical study of Compound 1 described in Example 1.

FIG. 7e illustrates the mean HDL profiles for placebo, Cohort 1 and Cohort 2 treatment groups after sMMTT performed as part of the clinical study of Compound 1 described in Example 1.

FIG. 7f illustrates the mean LDL profiles for placebo, Cohort 1 and Cohort 2 treatment groups after sMMTT performed as part of the clinical study of Compound 1 described in Example 1.

FIG. 7g illustrates the percent change from baseline (Day 1) for fasting triglycerides for placebo, Cohort 1 and Cohort 2 treatment groups in the clinical study of Compound 1 described in Example 1.

FIG. 7h illustrates the mean triglyceride profiles for placebo, Cohort 1 and Cohort 2 treatment groups after sMMTT performed as part of the clinical study of Compound 1 described in Example 1.

FIG. 8 illustrates the VAS appetite assessments—hunger, fullness and overall appetite—for the placebo, Cohort 1 and Cohort 2 treatment groups in the clinical study of Compound 1 described in Example 1.

### DETAILED DESCRIPTION

### Overview

A number of peptides derived from pre-proglucagon, and analogues thereof, have been proposed as therapeutics for the treatment of T2D and obesity, in particular, Gcg, GLP-1 and oxyntomodulin (OXM). These molecules are involved in a wide variety of physiological functions, including glucose homeostasis, insulin secretion, gastric emptying and intestinal growth, as well as regulation of food intake.

Gcg is a 29-amino acid peptide that corresponds to amino acids 53 to 81 of preproglucagon. OXM is a 37 amino acid peptide and is composed of the complete 29 amino acid sequence of Gcg with an octapeptide carboxy terminal extension (amino acids 82 to 89 of pre-proglucagon and termed “intervening peptide 1” or IP-1). Gcg helps maintain the level of glucose in the blood by binding to Gcg receptors on hepatocytes, causing the liver to release glucose-stored in the form of glycogen-through glycogenolysis. As these stores become depleted, Gcg stimulates the liver to synthesize additional glucose by gluconeogenesis. This glucose is released into the bloodstream, preventing the development of hypoglycemia.

GLP-1 has different biological activities compared to Gcg. Its actions include stimulation of insulin synthesis and secretion, inhibition of Gcg secretion and inhibition of food intake. GLP-1 has been shown to reduce hyperglycemia in diabetics. Several GLP-1 agonists have been approved for use in the treatment of T2D in humans, including exenatide, liraglutide, lixisenatide, albiglutide and dulaglutide. Such GLP-1 agonists are effective in glycemic control with favorable effects on weight without the risk of hypoglycemia. However, the weight loss is modest due to dose-dependent gastrointestinal side-effects.

OXM activates both the Gcg and GLP-1 receptors, with a slightly higher potency for the Gcg receptor over the GLP-1 receptor. It is less potent than native Gcg and GLP-1 on their respective receptors. Human Gcg is also capable of activating both receptors, albeit with a strong preference for the Gcg receptor over the GLP-1 receptor. GLP-1 is not capable of activating Gcg receptors. OXM is involved in regulation of food intake and body weight. It has been shown to suppress appetite and inhibit food intake in humans. International Patent Application Publication No. WO 2016/209707 A1 describes the structure, function, production and use of OXM analogues (GLP-1/Gcg co-agonists) that can be used for treating diabetes, obesity, obesity related comorbidities and other medical conditions. Of particular interest herein is the OXM analogue (GLP-1/Gcg co-agonist) described therein in Example 2, which is a fatty acid acylated, long-acting OXM analogue (GLP-1/Gcg co-agonist) (SEQ ID NO:1).

Unfortunately, many individuals with diabetes fail to reach their HbA1c goals and struggle with weight management, so new therapies and dosing regimens capable of providing additional glycemic control and/or weight loss are needed. As noted hereinabove, simply increasing the dose of a therapeutic agent may not necessarily be capable of achieving increased efficacy, as the efficacy may plateau at certain dose levels. Further, while increasing the dose of a therapeutic agent may, in some cases, be capable of achieving increased efficacy, increasing the dose of the therapeutic agent, especially one having activity at each of the GLP-1 and GCG receptors, also carries a risk of greater side effects, in particular gastrointestinal side effects.

The invention described herein provides doses and dosing regimens that overcome these challenges and surprisingly deliver substantially improved weight loss, improved body composition, robust glucose control, lipid lowering and/or insulin sensitization sparing beta-cell insulin secretory workload while at the same time exhibiting only mild or moderate adverse events.

As described herein an unexpectedly high weight loss (−19.4 and −20.6%) was achieved in a 20 week study in two separate cohorts which received doses of greater than about 6 mg to about 16 mg compared to those receiving placebo. Such magnitude of weight loss in such short time has not been previously reported for any GLP-1/Gcg co-agonists.

In particular, it has been surprisingly found that the doses and regimen described herein achieve substantial weight loss and improved body composition, as evidenced by, for example, reduced body weight, waist circumference, absolute and % fat body mass and increased % lean body mass. It has also surprisingly been found that the doses and regimen described herein achieve glycemic control, as evidenced by, for example, substantial reductions in HbA1c, fasting glucose, and glucose excursions on a Mixed Meal Tolerance Test (MMTT). It has also been surprisingly found that the doses and regimen described herein achieve improved insulin sensitivity, as evidenced by, for example, reduced fasting insulin and C-peptide, reduced insulin excursions on MMTT and reduced HOMA-IR. It has also been surprisingly found that the doses and regimen described herein achieve a reduction in appetite, as evidenced by, for example, decreased hunger and increased fullness. It has also been surprisingly found that the doses and regimen described herein achieve improvements in lipid profiles, as evidenced by, for example, reductions in fasting and sMMTT total cholesterol, triglycerides, LDL cholesterol and HDL cholesterol. It has also been surprisingly found that the doses and regimen described herein achieve Gcg-R target engagement, as evidenced by reductions in fasting and sMMTT glucagon levels.

Multiple aspects of dosing regimens for and methods of using GLP-1/Gcg co-agonist suitable for once-weekly dosing are described herein. In certain instances, the regimens and methods described herein include determining and administering an initial dose of such GLP-1/Gcg co-agonist. In other aspects, the regimens and methods described herein include determining and administering maximal or highest doses, including when and how to adjust doses.

In one aspect, the present invention provides a method for providing chronic weight management, improving glycemic control, and/or reducing LDL cholesterol or triglycerides, in a patient in need thereof, wherein said method comprises:
(1) administering a once weekly dose of a compound of the following formula:

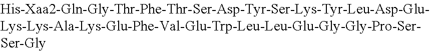

wherein Xaa2 is Aib;
Lys at position 20 is chemically modified by conjugation of the epsilon-amino group of the Lys side chain with ([2-(2-aminoethoxy)-ethoxy]-acetyl)₂-(γ-Glu)-CO—(CH₂)₁₈CO₂H; and
the C-terminal amino acid is amidated (SEQ ID NO:1),
or a pharmaceutically acceptable salt thereof,
to the patient in need thereof for at least about two weeks; and
(2) thereafter increasing the dose to greater than about 6 mg to about 16 mg and administering the increased dose to said patient once weekly for at least about two weeks.

In another aspect, provided herein is a method for preventing and/or treating a condition selected from type 2 diabetes, hyperglycemia, non-insulin dependent diabetes, obesity; dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, reducing body weight, reducing food intake, and/or inducing satiety in a patient, wherein said method comprises:
(1) administering a once weekly dose of a compound of SEQ ID NO:1, or a pharmaceutically acceptable salt thereof, to a patient in need thereof for at least about two weeks; and
(2) thereafter increasing the dose to greater than about 6 mg to about 16 mg and administering the increased dose to said patient once weekly for at least about two weeks.

In another aspect of the present disclosure, there is provided a compound of SEQ ID NO: 1, or a pharmaceutically acceptable salt thereof, for use in providing chronic weight management, improving glycemic control and/or reducing LDL cholesterol or triglycerides, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In another aspect, there is provided a compound of SEQ ID NO:1, or a pharmaceutically acceptable salt thereof, for use in preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, for use in reducing body weight, for use in reducing food intake and/or for use in inducing satiety in a patient, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In a further aspect of the present disclosure, there is provided the use of a compound of SEQ ID NO: 1, or a pharmaceutically acceptable salt thereof, in the manufacture of a medicament for providing chronic weight management, improving glycemic control, and/or reducing LDL cholesterol or triglycerides, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In another aspect, there is provided the use of a compound of SEQ ID NO:1, or a pharmaceutically acceptable salt thereof, in the manufacture of a medicament for preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, reducing body weight, reducing food intake and/or inducing satiety in a patient, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

In a further aspect of the present disclosure, the once weekly increased dose is from about 6.25 mg to about 16.0 mg, from about 6.5 mg to about 16.0 mg, from about 8.0 mg to about 16.0 mg, from about 10 mg to about 16.0 mg, from about 11.0 mg to about 16 mg, from about 12.0 mg to about 16.0 mg, from about 13.0 mg to about 16.0 mg or from about 14.0 mg to about 16.0.

In a further aspect of the present disclosure, the once weekly increased dose is about 6.25 mg, about 6.5 mg, about 7.0 mg, about 8.0 mg, about 9.0 mg, about 10.0 mg, about 11.0 mg, about 12.0 mg, about 13.0 mg, about 14.0 mg, about 15.0 mg or about 16.0 mg. In some embodiments, the once weekly increased dose is about 6.5 mg. In some embodiments, the once weekly increased dose is about 7.0 mg. In some embodiments, the once weekly increased dose is about 8.0 mg. In some embodiments, the once weekly increased dose is about 9.0 mg. In some embodiments, the once weekly increased dose is about 10.0 mg. In some embodiments, the once weekly increased dose is about 11.0 mg. In some embodiments, the once weekly increased dose is about 12.0 mg. In some embodiments, the once weekly increased dose is about 13.0 mg. In some embodiments, the once weekly increased dose is about 14.0 mg. In some embodiments, the once weekly increased dose is about 15.0 mg. In some embodiments, the once weekly increased dose is about 16.0 mg.

In a preferred aspect of the present disclosure, the once weekly increased dose is from about 8.0 mg to about 16.0 mg. In a still further aspect of the present disclosure, the once weekly increased dose is about 8.0 mg, about 10.0 mg, about 11.0 mg, about 12.0 mg, about 13.0 mg, about 14.0 mg, about 15.0 mg or about 16.0 mg.

In another preferred aspect of the present disclosure, the once weekly increased dose is from about 10.0 mg to about 16.0 mg. In a still further aspect of the present disclosure, the once weekly increased dose is about 10.0 mg, about 11.0 mg, about 12.0 mg, about 13.0 mg, about 14.0 mg, about 15.0 mg or about 16.0 mg.

In one aspect of the present disclosure, the once weekly dose in step (1) is about 1.0 mg to about 6.0 mg. In a still further aspect of the present disclosure, the about weekly dose is about 1.0 mg about 1.5 mg, about 2.0 mg, about 2.5 mg, about 3.0 mg, about 3.5 mg, about 4.0 mg, about 5.0 mg, about 5.5 mg or about 6.0 mg.

In a still further aspect of the present disclosure, multiple different once weekly doses are administered to the patient in steps (1) and (2).

In one aspect of the present disclosure, the patient is administered a first or initial once weekly dose of about 1.0 mg to about 6.0 mg. Thus, in some embodiments, a first once weekly dose is about 1.0 mg to about 6.0 mg. In a further aspect of the present disclosure, the first once weekly dose is about 1.0 mg, about 1.5 mg, about 2.0 mg, about 2.5 mg, about 3.0 mg, about 3.5 mg, about 4.0 mg, about 4.5 mg, about 5.0 mg, about 5.5 mg or about 6.0 mg. In a preferred aspect of the present disclosure, the first once weekly dose is about 1.0 mg, about 1.5 mg, about 2.0 mg or about 3.0 mg. In some embodiments, the first once weekly dose is about 1.0 mg. In some embodiments, the first once weekly dose is about 1.5 mg. In some embodiments, the first once weekly dose is about 2.0 mg. In some embodiments, the first once weekly dose is about 3.0 mg.

In one aspect of the present disclosure, the first dose and each subsequent increased dose are increased in increments of about 1.5 mg, about 2.0 mg, about 2.5 mg, about 3.0 mg, about 3.5 mg, about 4.0 mg, about 4.5 mg, about 5.0 mg, about 5.5 mg, about 6.0 mg or any combination thereof, and wherein each dose is administered for at least about two weeks. Thus, the first dose is selected from about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, and about 6.5 mg, and each subsequent dose is increased over the previously administered dose in an increment of about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg or any combination thereof.

In a preferred aspect, the first dose is selected from about 1.5 mg, about 2.0 mg, and about 3.0 mg and each subsequent dose is increased over the previously administered dose in an increment of about 1.5 mg, about 2.0 mg, about 3.0 mg, about 4.0 mg or any combination thereof. For instance, a patient is administered a first dose of 1.5 mg, a subsequent dose of 3.0 mg (an increment of 1.5 mg over the previous dose of 1.5 mg), a subsequent dose of 6.0 mg (an increment of 3.0 mg over the previous dose of 3.0 mg), a subsequent dose of 8.0 mg (an increment of 2.0 mg over the previous dose of 6.0 mg) and a subsequent dose of 10.0 mg (an increment of 2.0 mg over the previous dose of 8.0 mg). In another instance, a patient is administered a first dose of 1.5 mg, a subsequent dose of 3.0 mg (an increment of 1.5 mg over the previous dose of 1.5 mg), a subsequent dose of 6.0 mg (an increment of 3.0 mg over the previous dose of 3.0 mg), a subsequent dose of 10.0 mg (an increment of 4.0 mg over the previous dose of 6.0 mg), a subsequent dose of 13.0 mg (an increment of 3.0 mg over the previous dose of 10.0 mg) and a subsequent dose of 16.0 mg (an increment of 3.0 mg over the previous dose of 13.0 mg).

In another aspect of the present disclosure, the dose may be lowered after administering the maximal or highest dose for that patient. For instance, once weekly doses of 2.0 mg, 4.0 mg, 6.0 mg, 8.0 mg and 10.0 mg of the compound of SEQ ID NO:1, or a pharmaceutically acceptable salt thereof, are administered to the patient, each for three weeks. After three weeks at the maximal dose of 10 mg, the dose is reduced to 8 mg.

In another aspect of the present disclosure, the method or use comprises administering each once weekly dose for at least about four weeks. In a still further aspect of the present disclosure, the method or use comprises administering each once weekly dose for at least about six weeks. In a still further aspect of the present disclosure, the method or use comprises administering each once weekly dose for at least about eight weeks. Different once weekly doses may be administered for different time periods. For instance, the first dose and subsequent increased doses is administered for about four weeks and the maximal increased dose is administered for at least about eight weeks. In another instance, the first dose is administered for two weeks, each subsequent increased dose is administered for about two or four weeks and the maximal increased dose is administered for at least about eight weeks.

In a preferred aspect of the present disclosure, the maximal increased once weekly dose of the compound, or a pharmaceutically acceptable salt thereof, is about 6.5 mg.

In one embodiment, the method or use comprises:
a) administering a dose of about 3.0 mg of the compound, or a pharmaceutically acceptable salt thereof, once weekly for at least about two weeks, at least about 3 weeks, or at least about four weeks; and
b) increasing the dose to about 6.5 mg of the compound, or a pharmaceutically acceptable salt thereof, once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) optionally increasing the dose to about 4.5 mg of the compound, or a pharmaceutically acceptable salt thereof, and administering the dose once weekly for at least about two weeks, at least about three weeks, or at least about four weeks; and
(d) increasing the dose to about 6.5 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) increasing the dose to about 4.0 mg or about 5.0 mg of the compound, or a pharmaceutically acceptable salt thereof, and administering the dose once weekly for at least about two weeks, at least about three weeks, or at least about four weeks; and
(c) increasing the dose to about 6.5 mg of the compound, or a pharmaceutically acceptable salt thereof, and administering the dose once weekly for at least about two weeks, at least about four weeks, or at least about eight weeks.

In an alternative preferred aspect of the present disclosure, the maximal increased once weekly dose of the compound, or a pharmaceutically acceptable salt thereof, is about 8.0 mg.

In one embodiment, the method or use comprises:
a) administering a dose of about 4.0 mg of the compound, or a pharmaceutically acceptable salt thereof, once weekly for at least about two weeks, at least about three weeks, or at least about four weeks; and
b) increasing the dose to about 8.0 mg of the compound, or a pharmaceutically acceptable salt thereof, once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 6.0 mg of the compound, or a pharmaceutically acceptable salt thereof, and administering the dose once weekly for at least about two weeks, at least about three weeks, or at least about four weeks; and
(d) increasing the dose to about 8.0 mg of the compound, or a pharmaceutically acceptable salt thereof, and administering the dose once weekly for at least about two weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) increasing the dose to about 4.0 mg of the compound, or a pharmaceutically acceptable salt thereof, and administering the dose once weekly for at least about two weeks, at least about three weeks, or at least about four weeks;
(c) increasing the dose to about 6.0 mg of the compound, or a pharmaceutically acceptable salt thereof, and administering the dose to the patient once weekly for at least about two weeks, at least about three weeks, or at least about four weeks; and
(d) increasing the dose to about 8.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased once weekly dose of the compound, or a pharmaceutically acceptable salt thereof, is about 9.0 mg.

In one embodiment, the method or use comprises:
a) administering a dose of about 4.5 mg of the compound, or a pharmaceutically acceptable salt thereof, once weekly for at least about two weeks, at least about three weeks, or at least about four weeks; and
b) increasing the dose to about 9.0 mg of the compound, or a b) pharmaceutically acceptable salt thereof, once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 6.0 mg of the compound, or a pharmaceutically acceptable salt thereof, and administering the dose once weekly for at least about two weeks, at least about three weeks, or at least about four weeks; and
(d) increasing the dose to about 9.0 mg of the compound, or a pharmaceutically acceptable salt thereof, and administering the dose once weekly for at least about two weeks, at least about three weeks, or at least about four weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) increasing the dose to about 4.0 mg of the compound, or a pharmaceutically acceptable salt thereof, and administering the dose once weekly for at least about two weeks, at least about three weeks, or at least about four weeks;
(c) increasing the dose to about 6.0 mg of the compound, or a pharmaceutically acceptable salt thereof, and administering the dose to the patient once weekly for at least about two weeks, at least about three weeks, or at least about four weeks; and
(d) increasing the dose to about 9.0 mg of the compound, or a pharmaceutically acceptable salt thereof, and administering the dose once weekly for at least about two weeks, at least about four weeks, at least about six weeks or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks; and
(c) increasing the dose to about 9.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased once weekly dose of the compound, or a pharmaceutically acceptable salt thereof, is about 10.0 mg.

In one embodiment, the method or use comprises:
a) administering a dose of about 5.0 mg of the compound, or a pharmaceutically acceptable salt thereof, once weekly for at least about two weeks, at least about three weeks, or at least about four weeks; and
b) increasing the dose to about 10.0 mg of the compound, or a pharmaceutically acceptable salt thereof, once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 5.0 mg or to about 6.0 mg of the compound, or a pharmaceutically acceptable salt thereof, and administering the dose once weekly for at least about two weeks, at least about three weeks, or at least about four weeks;
(d) increasing the dose to about 8.0 mg of the compound, or a pharmaceutically acceptable salt thereof, and administering the dose once weekly for at least about two weeks, at least about three weeks, or at least about four weeks; and
(e) increasing the dose to about 10.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) increasing the dose to about 4.0 mg of the compound, or a pharmaceutically acceptable salt thereof, and administering the dose once weekly for at least about two weeks, at least about three weeks, or at least about four weeks;
(c) increasing the dose to about 6.0 mg of the compound, or a pharmaceutically acceptable salt thereof, and administering the dose to the patient once weekly for at least about two weeks, at least about three weeks, or at least about four weeks;
(d) optionally increasing the dose to about 8.0 mg of the compound, or a pharmaceutically acceptable salt thereof, and administering the dose to the patient once weekly for at least about two weeks, at least about three weeks, or at least about four weeks; and
(e) increasing the dose to about 10.0 mg of the compound, or a pharmaceutically acceptable salt thereof, and administering the dose once weekly for at least about two weeks, at least about four weeks, at least about six weeks or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks; and
(c) increasing the dose to about 10.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased dose of the compound, or a pharmaceutically acceptable salt thereof, is about 12.0 mg.

In one embodiment, the method or use comprises:
a) administering a dose of about 6.0 mg of the compound, or a pharmaceutically acceptable salt thereof, once weekly for at least about two weeks, at least about three weeks, or at least about four weeks; and
b) increasing the dose to about 12.0 mg of the compound, or a pharmaceutically acceptable salt thereof, once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 5.0 mg or to about 6.0 mg of the compound, or a pharmaceutically acceptable salt thereof, and administering the dose once weekly for at least about two weeks, at least about three weeks, or at least about four weeks;
(d) increasing the dose to about 8.0 mg of the compound, or a pharmaceutically acceptable salt thereof, and administering the dose once weekly for at least about two weeks, at least about three weeks, or at least about four weeks;
(e) optionally increasing the dose of about 10.0 mg of the compound, or a pharmaceutically acceptable salt thereof, and administering the dose once weekly for at least about two weeks, at least about three weeks, or at least about four weeks; and
(f) increasing the dose to about 12.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) increasing the dose to about 4.0 mg of the compound, or a pharmaceutically acceptable salt thereof, and administering the dose once weekly for at least about two weeks, at least about three weeks, or at least about four weeks;
(c) increasing the dose to about 6.0 mg of the compound, or a pharmaceutically acceptable salt thereof, and administering the dose to the patient once weekly for at least about two weeks, at least about three weeks, or at least about four weeks;
(d) increasing the dose to about 8.0 mg of the compound, or a pharmaceutically acceptable salt thereof, and administering the dose to the patient once weekly for at least about two weeks, at least about three weeks, or at least about four weeks;
(e) optionally increasing the dose to about 10.0 mg of the compound, or a pharmaceutically acceptable salt thereof, and administering the dose to the patient once weekly for at least about two weeks, at least about three weeks, or at least about four weeks; and
(f) increasing the dose to about 12.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks;
(c) increasing the dose to about 9.0 mg or to about 10.0 mg of the compound, or a pharmaceutically acceptable salt thereof, and administering the dose once weekly for at least about two weeks, at least about three weeks, or at least about four weeks; and
(d) increasing the dose to about 12.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased dose of the compound, or a pharmaceutically acceptable salt thereof, is about 13.0 mg.

In one embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 5.0 mg or to about 6.0 mg of the compound, or a pharmaceutically acceptable salt thereof, and administering the dose once weekly for at least about two weeks, at least about three weeks, or at least about four weeks;
(d) optionally increasing the dose to about 8.0 mg of the compound, or a pharmaceutically acceptable salt thereof, and administering the dose once weekly for at least about two weeks, at least about three weeks, or at least about four weeks;
(e) increasing the dose to about 9.0 mg or to about 10.0 mg of the compound, or a pharmaceutically acceptable salt thereof, and administering the dose once weekly for at least about two weeks, at least about three weeks, or at least about four weeks; and
(f) increasing the dose to about 13.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) optionally increasing the dose to about 3.0 mg of the compound, or a pharmaceutically acceptable salt thereof, and administering the dose once weekly for at least about two weeks, at least about three weeks, or at least about four weeks;
(c) increasing the dose to about 4.0 mg of the compound, or a pharmaceutically acceptable salt thereof, and administering the dose once weekly for at least about two weeks, at least about three weeks, or at least about four weeks;
(d) increasing the dose to about 6.0 mg of the compound, or a pharmaceutically acceptable salt thereof, and administering the dose to the patient once weekly for at least about two weeks, at least about three weeks, or at least about four weeks;
(e) optionally increasing the dose to about 8.0 mg of the compound, or a pharmaceutically acceptable salt thereof, and administering the dose to the patient once weekly for at least about two weeks, at least about three weeks, or at least about four weeks;
(f) increasing the dose to about 9.0 mg or to about 10.0 mg of the compound, or a pharmaceutically acceptable salt thereof, and administering the dose to the patient once weekly for at least about two weeks, at least about three weeks, or at least about four weeks; and
(g) increasing the dose to about 13.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks;
(c) increasing the dose to about 9.0 mg or to about 10.0 mg of the compound, or a pharmaceutically acceptable salt thereof, and administering the dose once weekly for at least about two weeks, at least about three weeks, or at least about four weeks;
(d) increasing the dose to about 13.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased dose of the compound, or a pharmaceutically acceptable salt thereof, is about 14.0 mg.

In one embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 5.0 mg or to about 6.0 mg of the compound, or a pharmaceutically acceptable salt thereof, and administering the dose once weekly for at least about two weeks, at least about three weeks, or at least about four weeks;
(d) increasing the dose to about 8.0 mg of the compound, or a pharmaceutically acceptable salt thereof, and administering the dose once weekly for at least about two weeks, at least about three weeks, or at least about four weeks;
(e) optionally increasing the dose to about 10.0 mg of the compound, or a pharmaceutically acceptable salt thereof, and administering the dose once weekly for at least about two weeks, at least about three weeks, or at least about four weeks;
(f) increasing the dose to about 11.0 mg or to about 12.0 mg of the compound, or a pharmaceutically acceptable salt thereof, and administering the dose once weekly for at least about two weeks, at least about three weeks, or at least about four weeks; and
(g) increasing the dose to about 14.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) optionally increasing the dose to about 3.0 mg of the compound, or a pharmaceutically acceptable salt thereof, and administering the dose once weekly for at least about two weeks, at least about three weeks, or at least about four weeks;
(c) increasing the dose to about 4.0 mg of the compound, or a pharmaceutically acceptable salt thereof, and administering the dose once weekly for at least about two weeks, at least about three weeks, or at least about four weeks;
(d) increasing the dose to about 6.0 mg of the compound, or a pharmaceutically acceptable salt thereof, and administering the dose to the patient once weekly for at least about two weeks, at least about three weeks, or at least about four weeks;
(e) optionally increasing the dose to about 8.0 mg of the compound, or a pharmaceutically acceptable salt thereof, and administering the dose to the patient once weekly for at least about two weeks, at least about three weeks, or at least about four weeks;
(f) increasing the dose to about 9.0 mg or to about 10.0 mg of the compound, or a pharmaceutically acceptable salt thereof, and administering the dose to the patient once weekly for at least about two weeks, at least about three weeks, or at least about four weeks;
(g) optionally increasing the dose to about 12.0 mg or to about 13.0 mg of the compound, or a pharmaceutically acceptable salt thereof, and administering the dose to the patient once weekly for at least about two weeks, at least about three weeks, or at least about four weeks; and
(h) increasing the dose to about 14.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks;
(c) increasing the dose to about 9.0 mg or to about 10.0 mg of the compound, or a pharmaceutically acceptable salt thereof, and administering the dose once weekly for at least about two weeks, at least about three weeks, or at least about four weeks;
(d) optionally increasing the dose to about 12.0 mg of the compound, or a pharmaceutically acceptable salt thereof, and administering the dose once weekly for at least about two weeks, at least about three weeks, or at least about four weeks; and
(e) increasing the dose to about 14.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased dose of the compound, or a pharmaceutically acceptable salt thereof, is about 15.0 mg.

In one embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 5.0 mg or to about 6.0 mg of the compound, or a pharmaceutically acceptable salt thereof, and administering the dose once weekly for at least about two weeks, at least about three weeks, or at least about four weeks;
(d) optionally increasing the dose to about 8.0 mg of the compound, or a pharmaceutically acceptable salt thereof, and administering the dose once weekly for at least about two weeks, at least about three weeks, or at least about four weeks;
(e) increasing the dose to about 10.0 mg of the compound, or a pharmaceutically acceptable salt thereof, and administering the dose once weekly for at least about two weeks, at least about three weeks, or at least about four weeks;
(f) optionally increasing the dose to about 13.0 mg of the compound, or a pharmaceutically acceptable salt thereof, and administering the dose once weekly for at least about two weeks, at least about three weeks, or at least about four weeks; and
(g) increasing the dose to about 15.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) optionally increasing the dose to about 3.0 mg of the compound, or a pharmaceutically acceptable salt thereof, and administering the dose once weekly for at least about two weeks, at least about three weeks, or at least about four weeks;
(c) increasing the dose to about 4.0 mg of the compound, or a pharmaceutically acceptable salt thereof, and administering the dose once weekly for at least about two weeks, at least about three weeks, or at least about four weeks;
(d) increasing the dose to about 6.0 mg of the compound, or a pharmaceutically acceptable salt thereof, and administering the dose to the patient once weekly for at least about two weeks, at least about three weeks, or at least about four weeks;
(e) optionally increasing the dose to about 8.0 mg of the compound, or a pharmaceutically acceptable salt thereof, and administering the dose to the patient once weekly for at least about two weeks, at least about three weeks, or at least about four weeks;
(f) increasing the dose to about 9.0 mg or to about 10.0 mg of the compound, or a pharmaceutically acceptable salt thereof, and administering the dose to the patient once weekly for at least about two weeks, at least about three weeks, or at least about four weeks;
(g) optionally increasing the dose to about 12.0 mg or to about 13.0 mg of the compound, or a pharmaceutically acceptable salt thereof, and administering the dose to the patient once weekly for at least about two weeks, at least about three weeks, or at least about four weeks; and
(h) increasing the dose to about 15.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks;
(c) increasing the dose to about 9.0 mg or to about 10.0 mg of the compound, or a pharmaceutically acceptable salt thereof, and administering the dose once weekly for at least about two weeks, at least about three weeks, or at least about four weeks;
(d) optionally increasing the dose to about 12.0 mg or to about 13.0 mg of the compound, or a pharmaceutically acceptable salt thereof, and administering the dose once weekly for at least about two weeks, at least about three weeks, or at least about four weeks; and
(e) increasing the dose to about 15.0 mg of the compound, or a pharmaceutically acceptable salt thereof, and administering the dose once weekly for at least about two weeks, at least about four weeks, at least about six weeks, or at least about eight weeks.

In an alternative preferred aspect of the present disclosure, the maximal increased dose of the compound, or a pharmaceutically acceptable salt thereof, is about 16.0 mg.

In one embodiment, the method or use comprises:
(a) administering a first dose of about 1.5 mg of the compound, or a pharmaceutically acceptable salt thereof, once weekly for at least about two weeks, at least about three weeks, or at least about four weeks;
(b) increasing the dose to about 3.0 mg of the compound, or a pharmaceutically acceptable salt thereof, and administering the dose once weekly for at least about two weeks, at least about three weeks, or at least about four weeks;
(c) increasing the dose to about 5.0 mg or to about 6.0 mg of the compound, or a pharmaceutically acceptable salt thereof, and administering the dose once weekly for at least about two weeks, at least about three weeks, or at least about four weeks;
(d) optionally increasing the dose to about 8.0 mg of the compound, or a pharmaceutically acceptable salt thereof, and administering the once weekly for at least about two weeks, at least about three weeks, or at least about four weeks;
(e) optionally increasing the dose to about 9.0 or 10.0 mg of the compound, or a pharmaceutically acceptable salt thereof, and administering the dose once weekly for at least about two weeks, at least about three weeks, or at least about four weeks;
(f) increasing the dose to about 12.0 mg or to about 13.0 mg of the compound, or a pharmaceutically acceptable salt thereof, and administering the dose once weekly for at least about two weeks, at least about three weeks, or at least about four weeks; and
(g) increasing the dose to about 16.0 mg of the compound, or a pharmaceutically acceptable salt thereof, and administering the dose once weekly for at least about two weeks, at least about four weeks, at least about 6 weeks, or at least about eight weeks.

In another embodiment, the method or use comprises:
(a) administering a first dose of about 2.0 mg of the compound, or a pharmaceutically acceptable salt thereof, once weekly for at least about two weeks, at least about three weeks, or at least about four weeks;
(b) optionally increasing the dose to about 3.0 mg of the compound, or a pharmaceutically acceptable salt thereof, and administering the dose once weekly for at least about two weeks, at least about three weeks, or at least about four weeks;
(c) increasing the dose to about 4.0 mg of the compound, or a pharmaceutically acceptable salt thereof, and administering the dose once weekly for at least about two weeks, at least about three weeks, or at least about four weeks;
(d) increasing the dose to about 6.0 mg of the compound, or a pharmaceutically acceptable salt thereof, and administering the dose to the patient once weekly for at least about two weeks, at least about three weeks, or at least about four weeks;
(e) optionally increasing the dose to about 8.0 mg of the compound, or a pharmaceutically acceptable salt thereof, and administering the dose to the patient once weekly for at least about two weeks, at least about three weeks, or at least about four weeks;
(f) increasing the dose to about 9.0 mg or to about 10.0 mg of the compound, or a pharmaceutically acceptable salt thereof, and administering the dose to the patient once weekly for at least about two weeks, at least about three weeks, or at least about four weeks;
(g) increasing the dose to about 12.0 mg or to about 13.0 mg of the compound, or a pharmaceutically acceptable salt thereof, and administering the dose to the patient once weekly for at least about two weeks, at least about three weeks, or at least about four weeks; and
(h) increasing the dose to about 16.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 3.0 mg of the compound, or a pharmaceutically acceptable salt thereof, once weekly for at least about two weeks, at least about three weeks, or at least about four weeks;
(b) increasing the dose to about 6.0 mg of the compound, or a pharmaceutically acceptable salt thereof, and administering the dose once weekly for at least about two weeks, at least about three weeks, or at least about four weeks;
(c) increasing the dose to about 9.0 mg or to about 10.0 mg of the compound, or a pharmaceutically acceptable salt thereof, and administering the dose once weekly for at least about two weeks, at least about three weeks, or at least about four weeks; and
(d) increasing the dose to about 12.0 mg or to about 13.0 mg of the compound, or a pharmaceutically acceptable salt thereof, and administering the dose once weekly for at least about two weeks, at least about three weeks, or at least about four weeks; and
(e) increasing the dose to about 16.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks; at least about three weeks, or at least about four weeks;
(b) increasing the dose to about 3.0 mg of the compound, or a pharmaceutically acceptable salt thereof, and administering the dose once weekly for at least about two weeks; at least about three weeks, or at least about four weeks;
(c) increasing the dose to about 6.0 mg of the compound, or a pharmaceutically acceptable salt thereof, and administering the dose once weekly for at least about two weeks; at least about three weeks, or at least about four weeks;
(d) increasing the dose to about 8.0 mg of the compound, or a pharmaceutically acceptable salt thereof, and administering the dose once weekly for at least about two weeks; at least about three weeks, or at least about four weeks;
(e) increasing the dose to about 12.0 mg of the compound, or a pharmaceutically acceptable salt thereof, and administering the dose once weekly for at least about two weeks; at least about three weeks, or at least about four weeks; and
(f) increasing the dose to about 16.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks; at least about three weeks, or at least about four weeks;
(b) increasing the dose to about 4.0 mg of the compound, or a pharmaceutically acceptable salt thereof, and administering the dose once weekly for at least about two weeks; at least about three weeks, or at least about four weeks;
(c) increasing the dose to about 6.0 mg of the compound, or a pharmaceutically acceptable salt thereof, and administering the dose once weekly for at least about two weeks; at least about three weeks, or at least about four weeks;
(d) increasing the dose to about 8.0 mg of the compound, or a pharmaceutically acceptable salt thereof, and administering the dose once weekly for at least about two weeks; at least about three weeks, or at least about four weeks;
(e) increasing the dose to about 10.0 mg of the compound, or a pharmaceutically acceptable salt thereof, and administering the dose once weekly for at least about two weeks; at least about three weeks, or at least about four weeks;
(f) increasing the dose to about 13.0 mg of the compound, or a pharmaceutically acceptable salt thereof, and administering the dose once weekly for at least about two weeks; at least about three weeks, or at least about four weeks; and
(g) increasing the dose to about 16.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks; at least about three weeks, or at least about four weeks;
(b) increasing the dose to about 3.0 mg of the compound, or a pharmaceutically acceptable salt thereof, and administering the dose once weekly for at least about two weeks; at least about three weeks, or at least about four weeks;
(c) increasing the dose to about 6.0 mg of the compound, or a pharmaceutically acceptable salt thereof, and administering the dose once weekly for at least about two weeks; at least about three weeks, or at least about four weeks;
(d) optionally increasing the dose to about 8.0 mg of the compound, or a pharmaceutically acceptable salt thereof, and administering the dose once weekly for at least about two weeks; at least about three weeks, or at least about four weeks;
(e) increasing the dose to about 10.0 mg of the compound, or a pharmaceutically acceptable salt thereof, and administering the dose once weekly for at least about two weeks; at least about three weeks, or at least about four weeks;
(f) increasing the dose to about 13.0 mg of the compound, or a pharmaceutically acceptable salt thereof, and administering the dose once weekly for at least about two weeks; at least about three weeks, or at least about four weeks; and
(g) increasing the dose to about 16.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 1.5 mg of the compound, or a pharmaceutically acceptable salt thereof, once weekly for at least about two weeks; at least about three weeks, or at least about four weeks;
(b) increasing the dose to about 3.0 mg of the compound, or a pharmaceutically acceptable salt thereof, and administering the dose once weekly for at least about two weeks; at least about three weeks, or at least about four weeks;
(c) increasing the dose to about 6.0 mg of the compound, or a pharmaceutically acceptable salt thereof, and administering the dose once weekly for at least about two weeks; at least about three weeks, or at least about four weeks;
(d) increasing the dose to about 9.0 mg of the compound, or a pharmaceutically acceptable salt thereof, and administering the dose once weekly for at least about two weeks; at least about three weeks, or at least about four weeks;
(e) increasing the dose to about 12.0 mg of the compound, or a pharmaceutically acceptable salt thereof, and administering the dose once weekly for at least about two weeks; at least about three weeks, or at least about four weeks; and
(f) increasing the dose to about 16.0 mg of the compound, or a pharmaceutically acceptable salt thereof, and administering the dose once weekly for at least about two weeks, at least about four weeks, at least about six weeks, or at least about eight weeks.

In another embodiment, the method or use comprises:
(a) administering a first dose of about 2.0 mg of the compound, or a pharmaceutically acceptable salt thereof, once weekly for at least about two weeks; at least about three weeks, or at least about four weeks;
(b) increasing the dose to about 4.0 mg of the compound, or a pharmaceutically acceptable salt thereof, and administering the dose once weekly for at least about two weeks; at least about three weeks, or at least about four weeks;
(c) increasing the dose to about 6.0 mg of the compound, or a pharmaceutically acceptable salt thereof, and administering the dose once weekly for at least about two weeks; at least about three weeks, or at least about four weeks;
(d) increasing the dose to about 8.0 mg of the compound, or a pharmaceutically acceptable salt thereof, and administering the dose once weekly for at least about two weeks; at least about three weeks, or at least about four weeks;
(e) increasing the dose to about 10.0 mg of the compound, or a pharmaceutically acceptable salt thereof, and administering the dose once weekly for at least about two weeks; at least about three weeks, or at least about four weeks; and
(f) increasing the dose to about 16.0 mg of the compound, or a pharmaceutically acceptable salt thereof, and administering the dose once weekly for at least about two weeks, at least about four weeks, at least about six weeks, or at least about eight weeks.

In one aspect, the method or use comprises administering each dose once weekly for at least about two weeks. In one aspect, the method or use comprises administering each dose once weekly for at least about four weeks. In one aspect, the method or use comprises administering each dose once weekly for at least about eight weeks.

In one preferred aspect, the method or use comprises administering each dose once weekly for at least about two weeks and administering the maximal dose once weekly for at least about four weeks. In another preferred aspect, the method or use comprises administering each dose once weekly for at least about 4 weeks and administering the maximal dose once weekly for at least about 4 weeks. In another preferred aspect, the method or use comprises administering each dose once weekly for at least about 4 weeks and administering the maximal dose once weekly for at least about 8 weeks.

In one aspect, the administration of the compound of SEQ ID NO:1, or a pharmaceutically acceptable salt thereof, results in a decrease in the patient's body weight. In some embodiments, the administration of the compound of SEQ ID NO:1, or a pharmaceutically acceptable salt thereof, results in at least about 10% decrease in the patient's body weight. In some embodiments, the administration of the compound of SEQ ID NO: 1, or a pharmaceutically acceptable salt thereof, results in at least about 15% decrease in the patient's body weight. In some embodiments, the administration of the compound of SEQ ID NO: 1, or a pharmaceutically acceptable salt thereof, results in at least about 20% decrease in the patient's body weight.

In one aspect, the administration of the compound of SEQ ID NO:1, or a pharmaceutically acceptable salt thereof, results in a decrease in the patient's HbA1c. In one aspect, the administration of the compound of SEQ ID NO:1, or a pharmaceutically acceptable salt thereof, results in a decrease in the patient's LDL cholesterol and/or triglycerides decreases. In one aspect, the administration of the compound of SEQ ID NO: 1, or a pharmaceutically acceptable salt thereof, does not result in unacceptable tolerability.

In one aspect, the patient is overweight. In another aspect, the patient is obese. In another aspect, the patient has type 2 diabetes.

In another aspect, the present invention provides a method for providing chronic weight management, improving glycemic control, and/or reducing LDL cholesterol or triglycerides, in a patient in need thereof, wherein said method comprises:
(1) administering a once weekly escalation dose of a compound of SEQ ID NO:1, or a pharmaceutically acceptable salt thereof, to the patient in need thereof for at least about two weeks; and
(2) thereafter administering a once weekly maintenance dose greater than about 6 mg to about 16 mg of said compound, or a pharmaceutically acceptable salt thereof, for at least about two weeks.

In some embodiments, provided herein is a method for preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, reducing body weight, reducing food intake, and/or inducing satiety in a patient, wherein said method comprises:
(1) administering a once weekly escalation dose of a compound of SEQ ID NO:1, or a pharmaceutically acceptable salt thereof, to the patient in need thereof for at least about two weeks; and
(2) thereafter administering a once weekly maintenance dose greater than about 6 mg to about 16 mg of the said compound, or a pharmaceutically acceptable salt thereof, for at least about two weeks.

In another aspect of the present disclosure, there is provided a compound of SEQ ID NO: 1, or a pharmaceutically acceptable salt thereof, for use in chronic weight management, improving glycemic control and/or reducing LDL cholesterol or triglycerides, wherein:
(1) a once weekly escalation dose of the compound of SEQ ID NO:1, or a pharmaceutically acceptable salt thereof, is administered for at least about two weeks; and
(2) thereafter a once weekly maintenance dose greater than about 6 mg to about 16 mg of the said compound, or a pharmaceutically acceptable salt thereof, is administered for at least about two weeks.

In another aspect, there is provided a compound of SEQ ID NO:1, or a pharmaceutically acceptable salt thereof, for use in preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, for use in reducing body weight, for use in reducing food intake and/or for use in inducing satiety in a patient, wherein:
(1) a once weekly escalation dose of the compound of SEQ ID NO:1, or a pharmaceutically acceptable salt thereof, is administered for at least about two weeks; and
(2) thereafter a once weekly maintenance dose greater than about 6 mg to about 16 mg of the said compound, or a pharmaceutically acceptable salt thereof, is administered for at least about two weeks.

In a further aspect of the present disclosure, there is provided the use of a compound of SEQ ID NO: 1, or a pharmaceutically acceptable salt thereof, in the manufacture of a medicament for chronic weight management, improving glycemic control, and/or reducing LDL cholesterol or triglycerides, wherein:
(1) a once weekly escalation dose of the compound of SEQ ID NO:1, or a pharmaceutically acceptable salt thereof, is administered for at least about two weeks; and
(2) thereafter a once weekly maintenance dose greater than about 6 mg to about 16 mg of the said compound, or a pharmaceutically acceptable salt thereof, is administered for at least about two weeks.

In another aspect, there is provided the use of a compound of SEQ ID NO:1, or a pharmaceutically acceptable salt thereof, in the manufacture of a medicament for preventing and/or treating a condition selected from type 2 diabetes, hyperglycemia, non-insulin dependent diabetes, obesity, dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease, reducing body weight, reducing food intake and/or inducing satiety in a patient, wherein:
(1) a once weekly escalation dose of the compound of SEQ ID NO:1, or a pharmaceutically acceptable salt thereof, is administered for at least about two weeks; and
(2) thereafter a once weekly maintenance dose greater than about 6 mg to about 16 mg of the said compound, or a pharmaceutically acceptable salt thereof, is administered for at least about two weeks.

In a further aspect of the present disclosure, the once weekly maintenance dose is from about 6.25 mg to about 16.0 mg, from about 6.5 mg to about 16.0 mg, from about 8.0 mg to about 16.0 mg, from about 10 mg to about 16.0 mg, from about 11.0 mg to about 16 mg, from about 12.0 mg to about 16.0 mg, from about 13.0 mg to about 16.0 mg or from about 14.0 mg to about 16.0 mg.

In a further aspect of the present disclosure, the once weekly maintenance dose 6.25 mg, about 6.5 mg, about 7.0 mg, about 8.0 mg, about 9.0 mg, about 10.0 mg, about 11.0 mg, about 12.0 mg, about 13.0 mg, about 14.0 mg, about 15.0 mg or about 16.0 mg.

In a further aspect of the present disclosure, the once weekly maintenance dose is from about 8.0 mg to about 16.0 mg. In a still further aspect of the present disclosure, the once weekly maintenance dose is about 8.0 mg, about 10.0 mg, about 11.0 mg, about 12.0 mg, about 13.0 mg, about 14.0 mg, about 15.0 mg or about 16.0 mg.

In a still further aspect of the present disclosure, the once weekly maintenance dose is from about 10.0 mg to about 16.0 mg. In a still further aspect of the present disclosure, the once weekly maintenance dose is about 10.0 mg, about 11.0 mg, about 12.0 mg, about 13.0 mg, about 14.0 mg, about 15.0 mg or about 16.0 mg.

In a still further aspect of the present disclosure, the method or use comprises administering the once weekly maintenance dose for at least about four weeks. In a still further aspect of the present disclosure, the method or use comprises administering the once weekly maintenance dose for at least about six weeks. In a still further aspect of the present disclosure, the method or use comprises administering the once weekly maintenance dose for at least about eight weeks.

In a still further aspect of the present disclosure, the once weekly escalation dose is about 1.0 mg to about 6.0 mg. In a still further aspect of the present disclosure, the once weekly escalation dose is about 1.0 mg about 1.5 mg, about 2.0 mg, about 2.5 mg, about 3.0 mg, about 3.5 mg, about 4.0 mg, about 5.0 mg, about 5.5 mg or about 6.0 mg.

In a still further aspect of the present disclosure, multiple once weekly escalation doses are administered to the patient in steps (1) and (2).

In a preferred aspect of the present disclosure, the first once weekly escalation dose is about 1.0 mg to about 6.0 mg. In a further aspect of the present disclosure, the first once weekly escalation dose is about 1.0 mg, about 1.5 mg, about 2.0 mg, about 2.5 mg, about 3.0 mg, about 3.5 mg, about 4.0 mg, about 4.5 mg, about 5.0 mg, about 5.5 mg or about 6.0 mg. In a preferred aspect of the present disclosure, the once weekly escalation dose is about 1.0 mg, about 1.5 mg, about 2.0 mg, or about 3.0 mg.

In a further preferred aspect of the present disclosure, the first and each subsequent once weekly escalation dose are increased in increments of about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, or any combination thereof, and wherein each once weekly escalation dose is administered for at least about two weeks.

In a further preferred aspect of the present disclosure, the final or maximal escalation dose is higher than the maintenance dose. For instance, once weekly escalation doses of 2.0 mg, 4.0 mg, 6.0 mg, 8.0 mg and 10.0 mg of the compound of SEQ ID NO:1, or a pharmaceutically acceptable salt, thereof, are administered to the patient, each for three weeks. After three weeks at the highest escalation dose of 10 mg, the maintenance dose is reduced to 8 mg.

In an alternative preferred aspect of the present disclosure, the maintenance dose of the compound, or a pharmaceutically acceptable salt thereof, is 6.5 mg.

Preferably, the method or use comprises:
a) administering an escalation dose of about 3.0 mg of the compound, or a pharmaceutically acceptable salt thereof, once weekly for at least two weeks, at least about 3 weeks, or at least about four weeks; and
b) administering a maintenance dose of about 6.5 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) optionally increasing the escalation dose to about 4.5 mg of the compound, or a pharmaceutically acceptable salt thereof, and administering the escalation dose once weekly for at least about two weeks, at least about three weeks, or at least about four weeks; and
(d) administering a maintenance dose of about 6.5 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) increasing the escalation dose to about 4.0 mg or about 5.0 mg of the compound, or a pharmaceutically acceptable salt thereof, and administering the escalation dose once weekly for at least about two weeks, at least about three weeks, or at least about four weeks; and
(c) administering a maintenance dose of about 6.5 mg of the compound, or a pharmaceutically acceptable salt thereof, once weekly for at least about two weeks, at least about four weeks, or at least about eight weeks.

In an alternative preferred aspect of the present disclosure, the maintenance dose of the compound, or a pharmaceutically acceptable salt thereof, is 8.0 mg.

Preferably, the method or use comprises:
a) administering an escalation dose of about 4.0 mg of the compound, or a pharmaceutically acceptable salt thereof, once weekly for at least about two weeks, at least about three weeks, or at least about four weeks; and
b) administering a maintenance dose of about 8.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 6.0 mg of the compound, or a pharmaceutically acceptable salt thereof, and administering the escalation dose once weekly for at least about two weeks, at least about three weeks, or at least about four weeks; and
(d) administering a maintenance dose of about 8.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) increasing the escalation dose to about 4.0 mg of the compound, or a pharmaceutically acceptable salt thereof, and administering the escalation dose once weekly for at least about two weeks, at least about three weeks, or at least about four weeks;
(c) increasing the escalation dose to about 6.0 mg of the compound, or a pharmaceutically acceptable salt thereof, and administering the escalation dose to the patient once weekly for at least about two weeks, at least about three weeks, or at least about four weeks; and
(d) administering a maintenance dose of about 8.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 9.0 mg.

Preferably, the method or use comprises:
a) administering an escalation dose of about 4.5 mg of the compound, or a pharmaceutically acceptable salt thereof, once weekly for at least about two weeks, at least about three weeks, or at least about four weeks; and
b) administering a maintenance dose of about 10.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 6.0 mg of the compound, or a pharmaceutically acceptable salt thereof, and administering the escalation dose once weekly for at least about two weeks, at least about three weeks, or at least about four weeks; and
(d) increasing the escalation dose to about 9.0 mg of the compound, or a pharmaceutically acceptable salt thereof, and administering the escalation dose once weekly for at least about two weeks, at least about three weeks, or at least about four weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) increasing the escalation dose to about 4.0 mg of the compound, or a pharmaceutically acceptable salt thereof, and administering the escalation dose once weekly for at least about two weeks, at least about three weeks, or at least about four weeks;
(c) increasing the escalation dose to about 6.0 mg of the compound, or a pharmaceutically acceptable salt thereof, and administering the escalation dose to the patient once weekly for at least about two weeks, at least about three weeks, or at least about four weeks; and
(d) increasing the escalation dose to about 9.0 mg of the compound, or a pharmaceutically acceptable salt thereof, and administering the escalation dose to the patient once weekly for at least about two weeks, at least about three weeks, or at least about four weeks.

Alternatively, preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a pharmaceutically acceptable salt thereof, and administering the escalation dose once weekly for at least about two weeks, at least about three weeks, or at least about four weeks; and
(c) administering a maintenance dose of about 9.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 10.0 mg.

Preferably, the method or use comprises:
a) administering an escalation dose of about 5.0 mg of the compound, or a pharmaceutically acceptable salt thereof, once weekly for at least about two weeks, at least about three weeks, or at least about four weeks; and
b) administering a maintenance dose of about 10.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 5.0 mg or to about 6.0 mg of the compound, or a pharmaceutically acceptable salt thereof, and administering the escalation dose once weekly for at least about two weeks, at least about three weeks, or at least about four weeks;
(d) increasing the escalation dose to about 8.0 mg of the compound, or a dose once weekly for at least about two weeks, at least about three weeks, or at least about four weeks; and
(e) administering a maintenance dose of about 10.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) increasing the escalation dose to about 4.0 mg of the compound, or a pharmaceutically acceptable salt thereof, and administering the escalation dose once weekly for at least about two weeks, at least about three weeks, or at least about four weeks;
(c) increasing the escalation dose to about 6.0 mg of the compound, or a pharmaceutically acceptable salt thereof, and administering the escalation dose to the patient once weekly for at least about two weeks, at least about three weeks, or at least about four weeks;
(d) increasing the escalation dose to about 8.0 mg of the compound, or a pharmaceutically acceptable salt thereof, and administering the escalation dose to the patient once weekly for at least about two weeks, at least about three weeks, or at least about four weeks; and
(e) administering a maintenance dose of about 10.0 mg of the compound, or a pharmaceutically acceptable salt thereof, once weekly for at least about two weeks, at least about four weeks, at least about six weeks or at least about eight weeks.

Alternatively, preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a dose once weekly for at least about two weeks, at least about three weeks, or at least about four weeks; and
(c) administering a maintenance dose of about 10.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 12.0 mg.

Preferably, the method or use comprises:
a) administering an escalation dose of about 6.0 mg of the compound, or a pharmaceutically acceptable salt thereof, once weekly for at least about two weeks, at least about three weeks, or at least about four weeks; and
b) administering a maintenance dose of about 12.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 5.0 mg or to about 6.0 mg of the compound, or a pharmaceutically acceptable salt thereof, and administering the escalation dose once weekly for at least about two weeks, at least about three weeks, or at least about four weeks;
(d) increasing the escalation dose to about 8.0 mg of the compound, or a dose once weekly for at least about two weeks, at least about three weeks, or at least about four weeks;
(e) increasing the escalation dose of about 10.0 mg of the compound, or a pharmaceutically acceptable salt thereof, and administering the escalation dose once weekly for at least about two weeks, at least about three weeks, or at least about four weeks; and
(f) administering a maintenance dose of about 12.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) increasing the escalation dose to about 4.0 mg of the compound, or a pharmaceutically acceptable salt thereof, and administering the escalation dose once weekly for at least about two weeks, at least about three weeks, or at least about four weeks;
(c) increasing the escalation dose to about 6.0 mg of the compound, or a pharmaceutically acceptable salt thereof, and administering the escalation dose to the patient once weekly for at least about two weeks, at least about three weeks, or at least about four weeks;
(d) increasing the escalation dose to about 8.0 mg of the compound, or a pharmaceutically acceptable salt thereof, and administering the escalation dose to the patient once weekly for at least about two weeks, at least about three weeks, or at least about four weeks;
(e) increasing the escalation dose to about 10.0 mg of the compound, or a pharmaceutically acceptable salt thereof, and administering the escalation dose to the patient once weekly for at least about two weeks, at least about three weeks, or at least about four weeks; and
(f) administering a maintenance dose of about 12.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a pharmaceutically acceptable salt thereof, and administering the escalation dose once weekly for at least about two weeks, at least about three weeks, or at least about four weeks;
(c) increasing the escalation dose to about 9.0 mg or to about 10.0 mg of the compound, or a pharmaceutically acceptable salt thereof, and administering the escalation dose once weekly for at least about two weeks, at least about three weeks, or at least about four weeks; and
(d) administering a maintenance dose of about 12.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 13.0 mg.

Preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 5.0 mg or to about 6.0 mg of the compound, or a pharmaceutically acceptable salt thereof, and administering the escalation dose once weekly for at least about two weeks, at least about three weeks, or at least about four weeks;
(d) optionally increasing the escalation dose to about 8.0 mg of the compound, or a pharmaceutically acceptable salt thereof, and administering the escalation dose once weekly for at least about two weeks, at least about three weeks, or at least about four weeks;
(e) increasing the escalation dose to about 9.0 mg or to about 10.0 mg of the compound, or a pharmaceutically acceptable salt thereof, and administering the escalation dose once weekly for at least about two weeks, at least about three weeks, or at least about four weeks; and
(f) administering a maintenance dose of about 13.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) optionally increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 4.0 mg of the compound, or a pharmaceutically acceptable salt thereof, and administering the escalation dose once weekly for at least about two weeks, at least about three weeks, or at least about four weeks;
(d) increasing the escalation dose to about 6.0 mg of the compound, or a pharmaceutically acceptable salt thereof, and administering the escalation dose to the patient once weekly for at least about two weeks, at least about three weeks, or at least about four weeks;
(e) optionally increasing the escalation dose to about 8.0 mg of the compound, or a pharmaceutically acceptable salt thereof, and administering the escalation dose to the patient once weekly for at least about two weeks, at least about three weeks, or at least about four weeks;
(f) increasing the escalation dose to about 9.0 mg or to about 10.0 mg of the compound, or a pharmaceutically acceptable salt thereof, and administering the escalation dose to the patient once weekly for at least about two weeks, at least about three weeks, or at least about four weeks; and
(g) administering a maintenance dose of about 13.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a pharmaceutically acceptable salt thereof, and administering the escalation dose once weekly for at least about two weeks, at least about three weeks, or at least about four weeks;
(c) increasing the escalation dose to about 9.0 mg or to about 10.0 mg of the compound, or a pharmaceutically acceptable salt thereof, and administering the escalation dose once weekly for at least about two weeks, at least about three weeks, or at least about four weeks;
(d) administering a maintenance dose of about 13.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 14.0 mg.

Alternatively preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a dose once weekly for at least about two weeks, at least about three weeks, or at least about four weeks;
(c) increasing the escalation dose to about 5.0 mg or to about 6.0 mg of the compound, or a pharmaceutically acceptable salt thereof, and administering the escalation dose once weekly for at least about two weeks, at least about three weeks, or at least about four weeks;
(d) increasing the escalation dose to about 8.0 mg of the compound, or a pharmaceutically acceptable salt thereof, and administering the escalation dose once weekly for at least about two weeks, at least about three weeks, or at least about four weeks;
(e) optionally increasing the escalation dose to about 10.0 mg of the compound, or a pharmaceutically acceptable salt thereof, and administering the escalation dose once weekly for at least about two weeks, at least about three weeks, or at least about four weeks;
(f) increasing the escalation dose to about 11.0 mg or to about 12.0 mg of the compound, or a pharmaceutically acceptable salt thereof, and administering the escalation dose once weekly for at least about two weeks, at least about three weeks, or at least about four weeks; and
(g) administering a maintenance dose of about 14.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) optionally increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 4.0 mg of the compound, or a dose once weekly for at least about two weeks, at least about three weeks, or at least about four weeks;
(d) increasing the escalation dose to about 6.0 mg of the compound, or a pharmaceutically acceptable salt thereof, and administering the escalation dose to the patient once weekly for at least about two weeks, at least about three weeks, or at least about four weeks;
(e) optionally increasing the escalation dose to about 8.0 mg of the compound, or a pharmaceutically acceptable salt thereof, and administering the escalation dose to the patient once weekly for at least about two weeks, at least about three weeks, or at least about four weeks;
(f) increasing the escalation dose to about 9.0 mg or to about 10.0 mg of the compound, or a pharmaceutically acceptable salt thereof, and administering the escalation dose to the patient once weekly for at least about two weeks, at least about three weeks, or at least about four weeks;
(g) optionally increasing the escalation dose to about 12.0 mg or to about 13.0 mg of the compound, or a pharmaceutically acceptable salt thereof, and administering the escalation dose to the patient once weekly for at least about two weeks, at least about three weeks, or at least about four weeks; and
(h) administering a maintenance dose of about 14.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a dose once weekly for at least about two weeks, at least about three weeks, or at least about four weeks;
(c) increasing the escalation dose to about 9.0 mg or to about 10.0 mg of the compound, or a pharmaceutically acceptable salt thereof, and administering the escalation dose once weekly for at least about two weeks, at least about three weeks, or at least about four weeks;
(d) optionally increasing the dose to about 12.0 mg of the compound, or a pharmaceutically acceptable salt thereof, and administering the dose once weekly for at least about two weeks; and
(e) administering a maintenance dose of about 14.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 15.0 mg.

Preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 5.0 mg or to about 6.0 mg of the compound, or a pharmaceutically acceptable salt thereof, and administering the escalation dose once weekly for at least about two weeks, at least about three weeks, or at least about four weeks;
(d) optionally increasing the escalation dose to about 8.0 mg of the compound, or a pharmaceutically acceptable salt thereof, and administering the escalation dose once weekly for at least about two weeks, at least about three weeks, or at least about four weeks;
(e) increasing the escalation dose to about 10.0 mg of the compound, or a dose once weekly for at least about two weeks, at least about three weeks, or at least about four weeks;
(f) optionally increasing the escalation dose to about 13.0 mg of the compound, or a pharmaceutically acceptable salt thereof, and administering the escalation dose once weekly for at least about two weeks, at least about three weeks, or at least about four weeks; and
(g) administering a maintenance dose of about 15.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) optionally increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 4.0 mg of the compound, or a pharmaceutically acceptable salt thereof, and administering the escalation dose once weekly for at least about two weeks, at least about three weeks, or at least about four weeks;
(d) increasing the escalation dose to about 6.0 mg of the compound, or a pharmaceutically acceptable salt thereof, and administering the escalation dose to the patient once weekly for at least about two weeks, at least about three weeks, or at least about four weeks;
(e) optionally increasing the escalation dose to about 8.0 mg of the compound, or a pharmaceutically acceptable salt thereof, and administering the escalation dose to the patient once weekly for at least about two weeks, at least about three weeks, or at least about four weeks;
(f) increasing the escalation dose to about 9.0 mg or to about 10.0 mg of the compound, or a pharmaceutically acceptable salt thereof, and administering the escalation dose to the patient once weekly for at least about two weeks, at least about three weeks, or at least about four weeks;
(g) optionally increasing the escalation dose to about 12.0 mg or to about 13.0 mg of the compound, or a pharmaceutically acceptable salt thereof, and administering the escalation dose to the patient once weekly for at least about two weeks, at least about three weeks, or at least about four weeks; and
(h) administering a maintenance dose of about 15.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a pharmaceutically acceptable salt thereof, and administering the escalation dose once weekly for at least about two weeks, at least about three weeks, or at least about four weeks;
(c) increasing the escalation dose to about 9.0 mg or to about 10.0 mg of the compound, or a pharmaceutically acceptable salt thereof, and administering the escalation dose once weekly for at least about two weeks, at least about three weeks, or at least about four weeks;
(d) optionally increasing the escalation dose to about 12.0 mg or to about 13.0 mg of the compound, or a pharmaceutically acceptable salt thereof, and administering the escalation dose once weekly for at least about two weeks, at least about three weeks, or at least about four weeks; and
(e) administering a maintenance dose of about 15.0 mg of the compound, or a pharmaceutically acceptable salt thereof, once weekly for at least about two weeks, at least about four weeks, at least about six weeks, or at least about eight weeks.

In an alternative preferred aspect of the present disclosure, the maintenance dose of the compound, or a pharmaceutically acceptable salt thereof, is 16.0 mg.

Preferably, the method or use comprises:
(a) administering an escalation dose of about 1.5 mg of the compound, or a pharmaceutically acceptable salt thereof, once weekly for at least about two weeks, at least about three weeks, or at least about four weeks;
(b) increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 5.0 mg or to about 6.0 mg of the compound, or a pharmaceutically acceptable salt thereof, and administering the escalation dose once weekly for at least about two weeks, at least about three weeks, or at least about four weeks;
(d) optionally increasing the escalation dose to about 8.0 mg of the compound, or a pharmaceutically acceptable salt thereof, and administering the escalation once weekly for at least about two weeks, at least about three weeks, or at least about four weeks;
(e) optionally increasing the escalation dose to about 9.0 or about 10.0 mg of the compound, or a pharmaceutically acceptable salt thereof, and administering the escalation dose once weekly for at least about two weeks, at least about three weeks, or at least about four weeks;
(f) increasing the escalation dose to about 12.0 mg or to about 13.0 mg of the compound, or a pharmaceutically acceptable salt thereof, and administering the escalation dose once weekly for at least about two weeks, at least about three weeks, or at least about four weeks; and
(g) administering a maintenance dose of about 16.0 mg of the compound, or a pharmaceutically acceptable salt thereof, once weekly for at least about two weeks, at least about four weeks, at least about 6 weeks, or at least about eight weeks.

Alternatively preferably, the method or use comprises:
(a) administering an escalation dose of about 2.0 mg of the compound, or a pharmaceutically acceptable salt thereof, once weekly for at least about two weeks, at least about three weeks, or at least about four weeks;
(b) optionally increasing the escalation dose to about 3.0 mg of the compound, or a pharmaceutically acceptable salt thereof, and administering the escalation dose once weekly for at least about two weeks, at least about three weeks, or at least about four weeks;
(c) increasing the escalation dose to about 4.0 mg of the compound, or a pharmaceutically acceptable salt thereof, and administering the escalation dose once weekly for at least about two weeks, at least about three weeks, or at least about four weeks;
(d) increasing the escalation dose to about 6.0 mg of the compound, or a pharmaceutically acceptable salt thereof, and administering the escalation dose to the patient once weekly for at least about two weeks, at least about three weeks, or at least about four weeks;
(e) optionally increasing the escalation dose to about 8.0 mg of the compound, or a pharmaceutically acceptable salt thereof, and administering the escalation dose to the patient once weekly for at least about two weeks, at least about three weeks, or at least about four weeks;
(f) increasing the escalation dose to about 9.0 mg or to about 10.0 mg of the compound, or a pharmaceutically acceptable salt thereof, and administering the escalation dose to the patient once weekly for at least about two weeks, at least about three weeks, or at least about four weeks;
(g) optionally increasing the escalation dose to about 12.0 mg or to about 13.0 mg of the compound, or a pharmaceutically acceptable salt thereof, and administering the escalation dose to the patient once weekly for at least about two weeks, at least about three weeks, or at least about four weeks; and
(h) administering a maintenance dose of about 16.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 3.0 mg of the compound, or a pharmaceutically acceptable salt thereof, once weekly for at least about two weeks, at least about three weeks, or at least about four weeks;
(b) increasing the escalation dose to about 6.0 mg of the compound, or a pharmaceutically acceptable salt thereof, and administering the escalation dose once weekly for at least about two weeks, at least about three weeks, or at least about four weeks;
(c) increasing the escalation dose to about 9.0 mg or to about 10.0 mg of the compound, or a pharmaceutically acceptable salt thereof, and administering the escalation dose once weekly for at least about two weeks, at least about three weeks, or at least about four weeks; and
(d) increasing the escalation dose to about 12.0 mg or to about 13.0 mg of the compound, or a pharmaceutically acceptable salt thereof, and administering the escalation dose once weekly for at least about two weeks, at least about three weeks, or at least about four weeks; and
(e) administering a maintenance dose of about 16.0 mg of the compound, or a pharmaceutically acceptable salt thereof, once weekly for at least about two weeks, at least about four weeks, at least about six weeks, or at least about eight weeks.

Alternatively preferably, the method or use comprises:
(a) administering an escalation dose of about 1.5 mg of the compound, or a pharmaceutically acceptable salt thereof, once weekly for at least about two weeks;
(b) increasing the escalation dose to about 3.0 mg of the compound, or a pharmaceutically acceptable salt thereof, and administering the escalation dose once weekly for at least about two weeks;
(c) increasing the escalation dose to about 6.0 mg of the compound, or a dose once weekly for at least about two weeks;
(d) increasing the escalation dose to about 8.0 mg of the compound, or a pharmaceutically acceptable salt thereof, and administering the escalation dose once weekly for at least about two weeks;
(e) increasing the escalation dose to about 12.0 mg of the compound, or a pharmaceutically acceptable salt thereof, and administering the escalation dose once weekly for at least about two weeks; and
(f) administering a maintenance dose of about 16.0 mg of the compound, or a pharmaceutically acceptable salt thereof, once weekly for at least about two weeks.

Alternatively preferably, the method or use comprises:
(a) administering an escalation dose of about 2.0 mg of the compound, or a pharmaceutically acceptable salt thereof, once weekly for at least about two weeks;
(b) increasing the escalation dose to about 4.0 mg of the compound, or a pharmaceutically acceptable salt thereof, and administering the escalation dose once weekly for at least about two weeks;
(c) increasing the escalation dose to about 6.0 mg of the compound, or a pharmaceutically acceptable salt thereof, and administering the escalation dose once weekly for at least about two weeks;
(d) increasing the escalation dose to about 8.0 mg of the compound, or a pharmaceutically acceptable salt thereof, and administering the escalation dose once weekly for at least about two weeks;
(e) increasing the escalation dose to about 10.0 mg of the compound, or a pharmaceutically acceptable salt thereof, and administering the escalation dose once weekly for at least about two weeks;
(f) increasing the escalation dose to about 13.0 mg of the compound, or a pharmaceutically acceptable salt thereof, and administering the escalation dose once weekly for at least about two weeks; and
(g) administering a maintenance dose of about 16.0 mg of the compound, or a pharmaceutically acceptable salt thereof, once weekly for at least about two weeks.

Alternatively preferably, the method or use comprises:
(a) administering an escalation dose of about 1.5 mg of the compound, or a pharmaceutically acceptable salt thereof, once weekly for at least about two weeks;
(b) increasing the escalation dose to about 3.0 mg of the compound, or a pharmaceutically acceptable salt thereof, and administering the escalation dose once weekly for at least about two weeks;
(c) increasing the escalation dose to about 6.0 mg of the compound, or a pharmaceutically acceptable salt thereof, and administering the escalation dose once weekly for at least about two weeks;
(d) increasing the escalation dose to about 10.0 mg of the compound, or a pharmaceutically acceptable salt thereof, and administering the escalation dose once weekly for at least about two weeks;
(e) increasing the escalation dose to about 13.0 mg of the compound, or a pharmaceutically acceptable salt thereof, and administering the escalation dose once weekly for at least about two weeks; and
(f) administering a maintenance dose of about 16.0 mg of the compound, or a pharmaceutically acceptable salt thereof, once weekly for at least about two weeks.

Alternatively preferably, the method or use comprises:
(a) administering an escalation dose of about 1.5 mg of the compound, or a pharmaceutically acceptable salt thereof, once weekly for at least about two weeks;
(b) increasing the escalation dose to about 3.0 mg of the compound, or a pharmaceutically acceptable salt thereof, and administering the escalation dose once weekly for at least about two weeks;
(c) increasing the escalation dose to about 6.0 mg of the compound, or a pharmaceutically acceptable salt thereof, and administering the escalation dose once weekly for at least about two weeks;
(d) increasing the escalation dose to about 9.0 mg of the compound, or a dose once weekly for at least about two weeks;
(e) increasing the escalation dose to about 12.0 mg of the compound, or a pharmaceutically acceptable salt thereof, and administering the escalation dose once weekly for at least about two weeks; and
(f) administering a maintenance dose of about 16.0 mg of the compound, or a pharmaceutically acceptable salt thereof, once weekly for at least about two weeks.

Alternatively preferably, the method or use comprises:
(a) administering an escalation dose of about 2.0 mg of the compound, or a pharmaceutically acceptable salt thereof, once weekly for at least about two weeks;
(b) increasing the escalation dose to about 4.0 mg of the compound, or a pharmaceutically acceptable salt thereof, and administering the escalation dose once weekly for at least about two weeks;
(c) increasing the escalation dose to about 6.0 mg of the compound, or a pharmaceutically acceptable salt thereof, and administering the escalation dose once weekly for at least about two weeks;
(d) increasing the escalation dose to about 8.0 mg of the compound, or a pharmaceutically acceptable salt thereof, and administering the escalation dose once weekly for at least about two weeks;
(e) increasing the escalation dose to about 10.0 mg of the compound, or a pharmaceutically acceptable salt thereof, and administering the escalation dose once weekly for at least about two weeks; and
(f) administering a maintenance dose of about 16.0 mg of the compound, or a pharmaceutically acceptable salt thereof, once weekly for at least about two weeks.

More preferably, the method or use comprises administering each escalation dose once weekly for at least about two weeks and administering the maintenance dose once weekly for at least about four weeks.

Further more preferably, the method or use comprises administering each escalation dose once weekly for at least about four weeks and administering the maintenance dose once weekly for at least about four weeks.

Further more preferably, the method or use comprises administering each escalation dose once weekly for at least about four weeks and administering the maintenance dose once weekly for at least about eight weeks.

Unless defined otherwise, all technical and scientific terms used herein have the same meaning as commonly understood by one of skill in the art to which the disclosure pertains. Although any methods and materials similar to or equivalent to those described herein can be used in the practice or testing of the GLP-1/Gcg co-agonist, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one”.

### Definitions

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of the GLP-1 or Gcg receptors, “activity,” “activate,” “activating” and the like means a capacity of a compound, such as the GLP-1/Gcg co-agonist described herein, to bind to and induce a response at the receptor(s), as measured using assays known in the art.

As used herein, “amino acid” means a molecule that, from a chemical standpoint, is characterized by containing one or more amine groups and one or more carboxylic acid groups and may contain other functional groups. As is known in the art, there is a set of twenty amino acids that are designated as standard amino acids and that are used as building blocks for most of the peptides/polypeptides/proteins produced by any living being.

As used herein, “analog” or “analogue” means a compound, such as a synthetic peptide or polypeptide, that activates a target receptor and that elicits at least one in vivo or in vitro effect elicited by a native receptor agonist.

As used herein, “dose” or “doses” means a quantity of a GLP-1/Gcg co-agonist for once weekly dosing that is administered to an individual in a discrete amount at a particular point in time. When used in connection with the terms dose, dosing, doses and the like, “adjustment” means a quantity of any decrease or increase to the dose administered the prior week. When used in connection with the terms dose, dosing, doses and the like, “regimen” means a set of guidelines for determining and administering one or more doses and/or adjustments thereto.

As used herein, “effective amount” means an amount, concentration or dose of one or more of the GLP-1/Gcg co-agonist herein, or a pharmaceutically acceptable salt thereof which, upon single or multiple dose administration to an individual in need thereof, provides a desired effect in such an individual under diagnosis or treatment (i.e., may produce a clinically measurable difference in a condition of the individual such as, for example, a reduction in blood glucose, a reduction in HbA1c, a reduction in weight or body fat and/or a change body composition). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the GLP-1/Gcg co-agonist administered, the mode of administration, the bioavailability characteristics of the preparation administered, the dose regimen selected, the use of concomitant medication, and other relevant circumstances.

As used herein, “fasting glucose” means a blood sugar level from a sample of blood taken after an individual fasts for at least about 8 hours.

As used herein, “glycemic control” means a or a reduction of an individual's HbA1c levels. Likewise, “improving” and/or “improved” glycemic control means reductions in HbA1c. Moreover, “in need of further” glycemic control means a need for reductions in HbA1c.

As used herein, “hemoglobin A1c” or “HbA1c” means glycated hemoglobin and its levels, which develop when hemoglobin joins with glucose in the blood. HbA1c levels are a commonly used measure of glycemic control in individuals with diabetes, with decreased HbA1c levels generally indicating improved glycemic control. In the context of the disclosure, the doses, regimens and methods here result in a decrease in HbA1c. In certain instances, the decrease in HbA1c is decreased relative to the HbA1c levels resulting from an existing treatment with the same or even a different GLP-1/Gcg co-agonist, including other GLP-1/Gcg co-agonists.

As used herein, “incretin analog” means a compound having structural similarities with, but multiple differences from, each of OXM, GLP-1 and GCG, especially human OXM, human GLP-1 and human GCG. The incretin analogs herein include amino acid sequences resulting in the compounds having affinity for and activity at each of the GLP-1 and GCG receptors (i.e., dual receptor agonist activity). Exemplary incretin analogs and sequences for human OXM, GLP-1 and GCG for use herein are described in Intl. Patent Application Publication No. WO 2016/209707 A1. Of particular use herein is the GLP-1/Gcg co-agonist described in Example 2 of Intl. Patent Application Publication No. WO 2016/209707 A1, which has the following sequence:

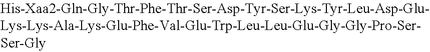

wherein Xaa2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH₂)₁₈CO₂H; and
the C-terminal amino acid is amidated (SEQ ID NO:1),
or a pharmaceutically acceptable salt thereof, including any protein that is the subject of a regulatory submission seeking approval of a GLP-1/Gcg co-agonist product that relies in whole or part upon data submitted to a regulatory agency by Eli Lilly and Company relating to this incretin analog, regardless of whether the party seeking approval of the product actually identifies the incretin analog as a GLP-1/Gcg co-agonist or uses some other term.

As used herein, “individual in need thereof” means a mammal, such as a human, with a condition, disease, disorder or symptom requiring treatment or therapy, including for example, those listed herein. In particular, the preferred individual to be treated is a human.

In certain aspects as used herein, “titration dose” or “escalation dose” means a dose that is less than the highest desired effective dose for the patient. As used herein, the disclosure contemplates that a “titration dose” or “escalation dose” may become the highest desired effective dose, or “maintenance dose” if such dose is observed to be the desired effective dose for the patient, and such dose may be administered chronically for a period exceeding at least two weeks. Alternatively, in certain aspects as used herein, “titration dose” or “escalation dose” means a dose that is higher than the maintenance dose for the patient. This aspect of the present disclosure is particularly useful in instances wherein the compound of SEQ ID NO: 1, or a pharmaceutically acceptable salt thereof, is administered, for example, to reduce body weight, to treat obesity, or to manage body weight. Significant weight loss may be obtained at the highest dose and the weight loss may be maintained at a lower dose.

As used herein “maintenance dose” means both a dose that is the highest desired effective dose for the patient, and the maintenance dose may become an escalation dose when such maintenance dose is less than the desired highest effective dose. That is to say, if, for a particular patient, the “about 8 mg” maintenance dose contemplated by the present disclosure is not the highest desired effective dose, then that 8 mg maintenance dose will become, in retrospect, a titration or escalation dose as the particular patient's dose will be increased up until reaching the next highest dose contemplated by the present disclosure, e.g., 10 mg for at least about 2 weeks or 12 mg for at least about 2 weeks. The disclosure contemplates that a patient that reaches a maintenance dose of about 12 mg to about 16 mg may, as determined by a physician or other health care provider, may need to have their dosage decreased to a lower maintenance dose.

As used herein, “expected weekly maintenance dose” means a dose of a therapeutic agent, such as a GLP-1/Gcg co-agonist, suitable for once-weekly dosing that would be expected to be needed to provide glycemic control or weight management in a given individual based on factors including, but not limited to, the individual's fasting glucose, HbA1c, frequency and severity of hypoglycemia and other AEs, and/or BW.

As used herein, “obese” or “obesity” means, with regard to an individual, one having a BMI ≥30 kg/m².

As used herein, “overweight” means, with regard to an individual, one having a BMI of >27 kg/m² but <30 kg/m².

As used herein, “treat,” “treating,” “to treat” and the like mean restraining, slowing, stopping or reversing the progression or severity of an existing condition, disease, disorder or symptom.

As used herein, and with reference to an incretin analog, “dual receptor agonist activity” means an incretin analog with activity at each of the GLP-1 and GCG receptors, especially an analog having a balanced and sufficient activity at each receptor to provide the benefits of agonism of that receptor while avoiding unwanted side effects associated with too much activity of that receptor. Moreover, the incretin analogs having dual receptor agonist activity have extended duration of action at each of the GLP-1 and GCG receptors, which advantageously allows for dosing as infrequently as once-a-day, thrice-weekly, twice-weekly or once-a-week.

As used herein, “weight management” means a reduction in body weight and/or change in body fat composition, as well as maintenance of body weight and/or body fat composition.

### Compositions

The compositions herein include a GLP-1/Gcg co-agonist having a structure of, for example, SEQ ID NO:1. The GLP-1/Gcg co-agonist can be synthetically produced (see, e.g., Intl. Patent Application Publication No. WO 2016/209707 A1). Formulations for the GLP-1/Gcg co-agonist that can be used herein are disclosed in Intl. Patent Application No. PCT/US2021/064592.

In some instances, the compositions herein are formulated to include a dose of the GLP-1/Gcg co-agonist from about 1.5 mg to about 16.0 mg. In other instances, the dose of the GLP-1/Gcg co-agonist can be about 1.5 mg, about 2.0 mg, about 3.0 mg, about 4.0 mg, about 4.5 mg, about 5.0 mg, about 6.0 mg, about 6.25 mg, about 6.5 mg, about 7.0 mg, about 8.0 mg, about 10.0 mg, about 12.0 mg, about 13.0 mg, about 14.0 mg, about 15.0 mg or about 16.0 mg. The compositions herein can be administered intravenously (IV), intramuscularly (IM) or subcutaneously (SC), especially SC. The compositions may be provided lyophilized and then reconstituted or as a solution formulation administered using a pre-filled, disposable pen, reusable pen, or automatic pen injector. Alternatively, the compositions may be administered using a multi-use vial or a pump device. In some instances, the device is an automatic injection apparatus as described in U.S. Pat. No. 8,734,394.

The compositions herein therefore may be presented in a pre-filled syringe/multi-use vial. Such pre-filled syringe/multi-use vial may be useful for administering about 0.5 mL to about 2.0 mL of the composition per patient per dose. The dose of the composition may be administered using a dosing schedule determined by a clinician, physician or other trained medical professional.

Alternatively, the composition can be prepared for a cartridge and therefore will differ from the above composition by including a preservative.

Alternatively, the composition can be prepared as part of an article of manufacture comprising the composition, where the article of manufacture can be a multi-use vial, a reusable pen injector, a pre-filled, disposable pen, an autoinjector or a pump.

In view of the above, the compositions herein are associated with acceptable shelf-life stability, in-use stability and acceptable injection site experience.

### Methods of Treatment and Medical Uses

The dosing regimen for the GLP-1/Gcg co-agonist described herein is suitable for providing chronic weight management. The dosing regimen for the GLP-1/Gcg co-agonist described herein is also suitable for improving glycemic control (as measured, e.g., by glucose levels, HbA1c, and/or fructosamine). The dosing regimen for the GLP-1/Gcg co-agonist described herein is also suitable for reducing LDL cholesterol and/or triglycerides.

The dosing regimen for the GLP-1/Gcg co-agonist described herein may be suitable for the prevention or treatment of a number of conditions, including but not limited to: prevention or treatment of type 2 diabetes mellitus, the prevention or treatment of hyperglycemia, the prevention of non-insulin dependent diabetes, the prevention or treatment of obesity, reduction of body weight and/or food intake, the prevention or treatment of dyslipidemia, the prevention or treatment of non-alcoholic fatty acid liver disease (NAFLD), the prevention or treatment of nonalcoholic steatohepatitis (NASH), the prevention of treatment of atherosclerosis, obstructive sleep opnea, osteoarthritis, polycystic ovary syndrome, chronic kidney disease and diabetic kidney disease. It is also suitable for inducing satiety, reducing fasting plasma glucose, reducing post-prandial blood glucose levels, reducing HbA1c, reducing adverse gastrointestinal events (e.g., nausea and vomiting), reducing systolic blood pressure, treating hypertension, reducing fructosamine levels, and improving of quality of life for subjects undergoing treatment.

In addition, the dosing regimen for the GLP-1/Gcg co-agonist described herein may be suitable for the following: treating obesity, controlling appetite, reducing caloric intake, reducing food intake, suppressing appetite, inducing anorexia, treating impaired glucose tolerance, treating post-prandial hyperglycemia, treating hyperglycemic conditions, reducing triglycerides, reducing cholesterol, treating impaired glucose tolerance, treating pre-diabetes (blood glucose levels that are higher than normal but not yet high enough to be diagnosed as diabetes), treating type 1 diabetes mellitus (e.g., in combination with insulin), reducing risk of a cardiovascular event due to impaired glucose tolerance, reducing risk of a cerebrovascular event due to impaired glucose tolerance, delaying the progression of diabetes, ameliorating diabetes, delaying diabetes onset, inducing β-cell preservation and restoring β-cell functionality, restoring normoglycemia, providing euglycemic control, treating gestational diabetes, treating or preventing nephropathy, treating or preventing cardiovascular diseases (e.g., heart failure, atherosclerosis, and acute coronary syndrome), treating or preventing metabolic syndrome.

In preferred embodiments, the dosing regimen for the GLP-1/Gcg co-agonist described herein is suitable for the following: providing chronic weight management, improving glycemic control, reducing LDL cholesterol and/or triglycerides, reducing body weight, reducing food intake, and/or inducing satiety. In some preferred embodiments, the dosing regimen for the GLP-1/Gcg co-agonist described herein is suitable preventing or treating a condition selected from type 2 diabetes, hyperglycemia, non-insulin dependent diabetes, obesity; dyslipidemia, non-alcoholic fatty acid liver disease (NAFLD), nonalcoholic steatohepatitis (NASH), major adverse cardiac events (MACE), obstructive sleep apnea, osteoarthritis, polycystic ovary syndrome, chronic kidney disease or diabetic kidney disease.

The following non-limiting examples are offered for purposes of illustration, not limitation.

### EXAMPLES

### Example 1: Clinical Study

### Overview

This study is a Phase 1, single-site, patient- and investigator-blinded, placebo controlled, randomized, non-crossover, multiple-ascending dose study with titration in each cohort in patients with T2DM to investigate the safety, tolerability, PK, and PD of the GLP-1/glucagon co-agonist peptide of SEQ ID NO:1 (Compound 1) administered as multiple SC injections QW for 12 weeks in Cohort 1 and 16 weeks in Cohort 2.

### Study Population

Investigators enrolled participants with T2DM for at least 6 months before screening into this study.

### Inclusion

The following patients were deemed eligible for participation in the study:
Otherwise generally healthy without known possible secondary diseases of diabetes mellitus.
Have a glycated haemoglobin (HbA1c) value at screening of >=7.0% and <=10.5% and treated with diet and exercise alone or a stable dose of metformin for at least 3 months prior to screening/Visit 1

### Exclusion

The following patients were deemed ineligible for participation in the study:
Have type 1 diabetes mellitus or latent autoimmune diabetes in adults.
Have uncontrolled diabetes defined as an episode of ketoacidosis or hyperosmolar state requiring hospitalization for 6 months prior to screening.
Have had an episode of severe hypoglycemia or have a history of hypoglycemia unawareness or poor recognition of hypoglycemic symptoms.

### Treatment

Two planned dose regimens of Compound 1 are investigated via weekly administration by SC injection in this study. The study is patient- and investigator-blinded. To preserve the blinding of the study for Compound 1 and placebo all study site personnel, except pharmacy staff who prepare and dispense study medication, are blinded to treatment allocation. Blinding of Compound 1 and placebo are maintained throughout the conduct of the trial until all data are assessed to an acceptable level of quality and locked.

The first dosing regimen (also referred to as Cohort 1) is 12 weeks in duration and requires dose increments of 1.5 mg starting from 1.5 mg:
1.5 mg QW Compound 1 for 4 weeks;
3.0 mg QW Compound 1 for 4 weeks; and
4.5 mg QW Compound 1 for 4 weeks.

The second dosing regimen (also referred to as Cohort 2) is 16 weeks in duration and requires dose increments of 2 mg starting from 2 mg:
2 mg QW Compound 1 for 3 weeks;
4 mg QW Compound 1 for 3 weeks;
6 mg QW Compound 1 for 3 weeks;
8 mg QW Compound 1 for 3 weeks; and
10 mg QW Compound 1 for 4 weeks.

In respect of the second dosing regimen, dose to 10 mg QW can only proceed if patients first tolerate titrations steps at 4 mg QW for 3 doses, 6 mg QW for 3 doses, then 8 mg QW for 3 doses.

In respect of each dosing regimen, Compound 1 is administered SC into the abdomen of the patient after an overnight fast for at least 8 hours.

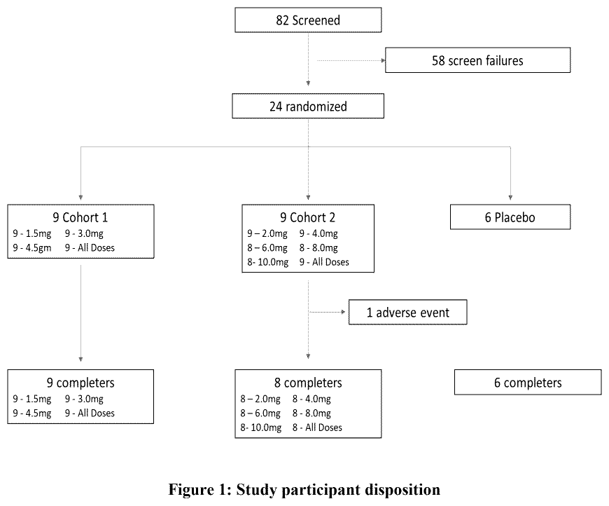

Of the 82 patients who gave informed consent and were screened in the study, 24 were randomly assigned and received at least 1 dose of study intervention. A total of 23 patients completed the study, as shown in FIG. 1.

### Dose Modification

Dose levels or increments, sampling schedule, and length of in the clinical research unit (CRU) stay may be adjusted in view of emerging safety, tolerability, or PK data during the study. In particular, dose increments within a cohort may be reduced (but not increased) or a lower dose may be administered, accordingly.

Actual doses, increments and/or duration of dosing within each cohort at each dose level may be adjusted in view of emerging safety, tolerability or PK data. By nature of being a multiple-ascending dose study with dose titration, data will be evaluated on an ongoing basis until the MTD is determined or stopping criteria have been met.

Safety data will be the primary criteria for the dose up-titration. No dose up-titration within a cohort can occur without prior discussion and agreement between the investigator and the appointed clinical pharmacologist. All safety and available pharmacokinetic (PK) and pharmacodynamic (PD) data one week prior to dose will be used to support dose within cohorts. All safety and available PK and PD data up to Week 11 will be used to support dose between Cohorts 1 and 2.

After review of these data, dose up-titration within a cohort to the next dose level will be made by the investigator and sponsor. If half of the patients in any cohort are unable to tolerate the dose titration, the dose increment could be halved and dose up-titration will occur every 2 weeks.

If any of the following stopping criteria occur in either cohort, dosing at the current level and further dose up-titration within both cohorts will be discontinued:
One treatment-emergent SAE (unless due to anticipated pharmacology of Compound 1 [e.g., hypoglycemia]) or two clinically significant events (CSEs) deemed possibly related to Compound 1 are reported, or
>=40% of subjects in a dose level experience a symptomatic-hypoglycemic episode with blood glucose values <=2.8 mmol/L (50 mg/dL; corresponding to plasma glucose [PG] levels of <=3.1 mmol/L [56 mg/dL]) and these events are deemed to be related to Compound 1 administration occur, or
Two or more subjects on active drug develop persistent (>1 week) symptoms suggestive of acute pancreatitis.
Two or more subjects with ‘severe’ non-serious adverse reactions, possibly related to the study drug, in the same cohort, independent of within or not within the same system organ class.

If any of these three scenarios occurs, dosing will be interrupted and will resume only if approved by the investigator or suitable designee.

### Study Assessments

The relevant study procedures and their timing are summarized below.

### Body Weight

In respect of Cohort 1, body weight is measured at baseline and Days 1, 8, 15, 22, 29, 36, 43, 50, 57, 64, 71 and 78.

In respect of Cohort 2, body weight is measured at baseline and Days 1, 8, 15, 22, 29, 36, 43, 50, 57, 64, 71, 78, 85, 92, 99, 106 and 109.

Wherever possible, the same scale is used for all weight measurements throughout the study and the scale is not moved or recalibrated. Subjects are weighed in light clothing at approximately the same time in the morning before dosing (on Day 1 only) and after an overnight fast and evacuation of bowel and the bladder, if possible. During the treatment period, weight is measured twice on each scheduled occasion, with the subject stepping off the scale between measurements. The average of the two weight measurements is recorded in the source document.

### Waist Circumference

In respect of Cohort 1, waist circumference is measured at baseline and Days 1, 8, 15, 22, 29, 36, 43, 50, 57, 64, 71 and 78.

In respect of Cohort 2, waist circumference is measured at baseline and Days 1, 8, 15, 22, 29, 36, 43, 50, 57, 64, 71, 78, 85, 92, 99, 106 and 109.

Waist circumference is measured at the midpoint between the lower margin of the least palpable rib and the top of the iliac crest. The patient stands with feet close together, arms at the side and body weight evenly distributed, and should wear little clothing. The patient should be relaxed, and the measurements should be taken at the end of a normal expiration. During screening, baseline, the treatment period, and at follow-up, waist circumference will be measured twice on each scheduled occasion. The average of the two measurements will be recorded in the source document.

### Pharmacokinetics (PK)

Venous blood samples of approximately 3 mL each are collected to determine the plasma concentrations of Compound 1. Up to two samples may be collected at additional time points during the study if warranted and agreed upon between both the investigator and sponsor. The actual date and time (24-hour clock time) of each sampling is recorded, as well as the date and time of each dose of Compound 1.

Drug concentration information that may unblind the study will not be reported to investigative sites or blinded personnel until the study has been unblinded.

In respect of Cohort 1, PK sampling occurs at Days 1 (12 hours post administration of Compound 1), 2 (24 hours post administration of Compound 1), 3 (48 hours post administration of Compound 1), 4 (72 hours post administration of Compound 1), 8, 22, 29, 31 (48 hours post administration of Compound 1), 50, 57, 59 (48 hours post administration of Compound 1), 78 (12 hours post administration of Compound 1), 79 (24 hours post administration of Compound 1), 80 (48 hours post administration of Compound 1) and 81 (72 hours post administration of Compound 1).

In respect of Cohort 2, PK sampling occurs at Days 1 (12 hours post administration of Compound 1), 2, (48 hours post administration of Compound 1), 72 (hours post administration of Compound 1), 8, 22, 24 (48 hours post administration of Compound 1), 36, 43, 45 (48 hours post administration of Compound 1), 64, 66 (48 hours post administration of Compound 1), 85, 87 (48 hours post administration of Compound 1), 92, 99, 106 (12 hours post administration of Compound 1), 107 (24 hours post administration of Compound 1), 108 (48 hours post administration of Compound 1) and 109 (72 hours post administration of Compound 1).

### HbA1c

In respect of Cohort 1, HbA1c levels are determined at baseline and Days 1, 29, 57 and 78. In respect of Cohort 2, HbA1c is measured at baseline and Days 1, 22, 43, 64, 85 and 106.

### Pharmacodynamics (PD)

Fasting plasma samples for glucose and insulin are explored as secondary PD markers. Exploratory PD may include, but are not limited to plasma glucagon, C-peptide, native OXM, lipid panel (triglycerides, total cholesterol, low-density lipoprotein cholesterol, and high-density lipoprotein cholesterol), beta cell function, and homeostatic model assessment (HOMA) indices.

Plasma concentrations of at least glucose, glucagon, insulin, C-peptide and native OXM, are assayed using validated analytical methods.

The samples are identified by the patient number (coded) and stored for up to a maximum of 1 year after the last patient visit for the study.

In respect of Cohort 1, PD sampling occurs at baseline and Days 1, 3 (48 hours post administration of Compound 1), 8, 22, 29, 50, 57, 78 and 80.

In respect of Cohort 2, PD sampling occurs at baseline and Days 1, 3 (48 hours post administration of Compound 1), 8, 22, 43, 64, 85, 106 and 108.

### Mixed Meal Tolerance Test (MMTT)

In respect of Cohort 1, the MMTT is performed at baseline and on Day 80. In respect of Cohort 2, the MMTT is performed at baseline and on Day 108.

Patients are provided breakfast meals for the MMTTs. Upon awakening, patients consume water to stay hydrated in order to mitigate problems with blood sample collections. Patients are provided individualized breakfast meals for the MMTTs. The total caloric content is approximately 30% of the daily estimated caloric need for weight. The macronutrient composition of the meals is targeted to provide approximately 50% of the calories from carbohydrate, 30% of the calories from fat, and 20% of the calories from protein. Patients are fasted (except for water) for at least 8 hours before each test meal and consume each meal within approximately 20 minutes. Test meals for each patient are kept consistent with respect to calorie and nutrient content across all MMTT assessments in the study.

For both parts of the study, the patient is not allowed to consume water (apart from fluid provided with the meal) for approximately 3 hours post MMTT until BOD POD® measurement.

While resident in the CRU, patients may not consume any food or caloric drinks other than that provided by the CRU. When not resident in the CRU, patients may resume their regular diet. The actual time of meal start and completion, total calories, and amount of meal (%) consumed in the MMTT are recorded.

### Appetite Analysis

To explore the effects of Compound 1 on meal intake and appetite sensation, subjects are asked to rate their appetite sensations using a 100-mm visual analogue scale

(VAS) for parameters of hunger, fullness, satiety, and prospective food consumption in the fasted state while inpatient as well as on scheduled outpatient visits. The VAS is a validated tool to assess appetite sensation parameters (Flint et al., Int J Obes Relat Metab Disord., 2000; 24(1):38-48). The VAS is presented as a 10-cm (100-mm) line, anchored by verbal descriptors, usually “extremely” and “not at all.” Subjects are required to rate their subjective sensations on four 100-mm scales combined with questions similar to the following:
“How hungry do you feel?”
“How satisfied do you feel?”
“How full do you feel?”
“How much do you think you could eat?”

Overall appetite score is calculated as the average of the 4 individual scores: satiety plus fullness plus (100−prospective food consumption) plus (100−hunger)/4 (van Can et al., Int J Obes (Lond). 2014; 38(6):784-793). The higher overall appetite score indicates less appetite, and the lower score indicates more appetite.

In respect of Cohort 1, the satiety VAS (after fasting) is completed at baseline and Days 1, 29, 57, 78 and 80.

In respect of Cohort 1, the satiety VAS (after fasting) is completed at baseline and Days 1, 22, 43, 64, 85, 106 and 108.

When the satiety VAS assessment coincides with the MMTT, it is performed pre-meal and 1 hour and 4 hours post MMTT.

### Gastric Emptying

Acetaminophen is a well-established marker for the rate and extent of gastric emptying. It is rapidly absorbed from the duodenum upon release from the stomach. A delay in gastric emptying is reflected in the alterations to the concentration-time profile of acetaminophen, specifically, decreasing its Cmax and increasing time of maximum observed drug concentration (tmax) without altering the extent (total drug amount) absorbed. An oral solution dose of approximately 1 g acetaminophen is considered to be sufficient for bioanalytical detection and is administered as indicated below in respect of Cohorts 1 and 2. Venous blood samples of approximately 2 mL each are collected to determine the plasma concentrations of acetaminophen. Concentrations of acetaminophen are assayed using a validated LC/MS method. Bioanalytical samples collected to measure acetaminophen concentrations are retained for a maximum of 2 years following last patient visit for the study.

In respect of Cohort 1, gastric emptying is assessed at baseline and Days 2 and 79.

In respect of Cohort 2, gastric emptying is assessed at baseline and Days 2 and 107.

### Adverse Events (AEs)

Investigators are responsible for monitoring the safety of patients who have entered this study. The investigator is responsible for the appropriate medical care of patients during the study. Investigators document their review of each laboratory safety report. The investigator remains responsible for following, through an appropriate health care option,

AEs that are serious or otherwise medically important, considered related to Compound 1 or the study, or that caused the patient to discontinue Compound 1 before completing the study. The patient is followed until the event resolves, stabilizes with appropriate diagnostic evaluation, or is reasonably explained. The frequency of follow-up evaluations of the AE is left to the discretion of the investigator.

The investigator records all relevant AE and SAE information. After receiving consent from the patient, study site personnel record the occurrence and nature of each patient's preexisting conditions, including clinically significant signs and symptoms of the disease under treatment in the study. Additionally, site personnel record any change in the condition(s) and the occurrence and nature of any AEs.

The investigator interprets and document whether or not an AE has a reasonable possibility of being related to study treatment, or a study procedure, taking into account the disease, concomitant treatment, or pathologies.

A “reasonable possibility” means that there is a potential cause and effect relationship between Compound 1, study device, and/or study procedure and the AE. Planned surgeries should not be reported as AEs unless the underlying medical condition has worsened during the course of the study.

A Serious Adverse Event (SAE) is any AE from this study that results in one of the following:
death
initial or prolonged inpatient hospitalization
a life-threatening experience (i.e., immediate risk of dying)
persistent or significant disability/incapacity
congenital anomaly/birth defect
important medical events that may not be immediately life-threatening or result in death or hospitalization but may jeopardize the patient or may require intervention to prevent one of the other outcomes listed in the definition above.

All AEs occurring after signing the ICF are recorded and assessed for serious criteria.

### Laboratory Tests

In respect of Cohort 1, samples for clinical laboratory tests are taken at baseline and at Days 1, 15, 29, 43, 57 and 78.

In respect of Cohort 2, samples for clinical laboratory tests are taken at baseline and at Days 1, 22, 43, 64, 85 and 106.

### Results

### Demographic and Other Baseline Characteristics

The baseline characteristics for patients with Type 2 diabetes are shown in Table 1. A total of 24 patients, 21 males and 3 females, between the ages of 40 and 68 years, inclusive, participated in this study.

TABLE 1
Summary of Baseline Characteristics (mean ± SD)

| | Compound 1 | Compound 1 | |
|---|---|---|---|
| Patient | Cohort 1 | Cohort 2 | Placebo |
| Characteristic | (n = 9) | (n = 9) | (n = 6) |
| Age (years) | 55.7 ± 8.0 | 61.4 ± 4.8 | 56.5 ± 10.1 |
| Weight (kg) | 97.5 ± 10.2 | 94.8 ± 19.9 | 94.7 ± 12.3 |
| Sex (male:female) | 8:1 | 7:2 | 6:0 |
| HbA1c (%) | 8.5 ± 1.0 | 8.1 ± 0.7 | 7.7 ± 0.7 |
| Fasting glucose (mmol/L) | 10.6 ± 2.2 | 10.8 ± 2.4 | 9.6 ± 1.9 |

### Concomitant and Post-Intervention Therapy

The current diabetes treatment during the study is summarized in Table 2.

TABLE 2
Summary of Current Diabetes Treatment

| | Treatment Group | | | |
|---|---|---|---|---|
| Ongoing | | Compound 1 | Compound 1 | Placebo |
| Diabetes | | Cohort 1 | Cohort 2 | (pooled) |
| Medication | | (n = 9) | (n = 9) | (n = 6) |

| | | | | | | |
|---|---|---|---|---|---|---|
| Sodium-Glucose | 0 | (0%) | 0 | (0%) | 1 | (16.7%) |
| Co-transporter 2 | | | | | | |
| inhibitors) | | | | | | |
| Biguanides | 0 | (0%) | 0 | (0%) | 1 | (16.7%) |
| Metformin | 8 | (88.9%) | 9 | (100%) | 6 | (100%) |

Abbreviations: n = number of patients.

### Body Weight

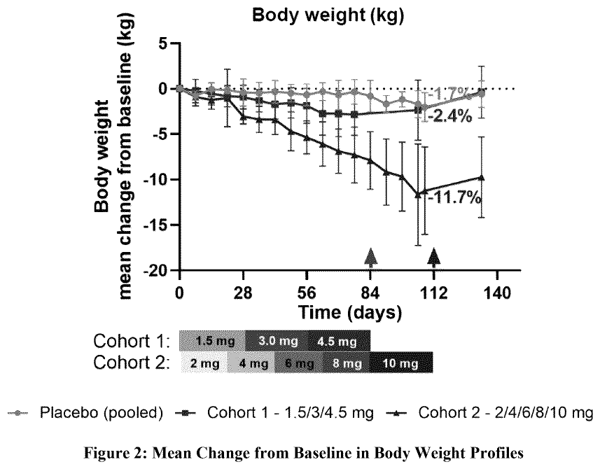

The body weight mean change from baseline is shown in FIG. 2. The mean body weight changes from baseline ranged from ranged from −2.30 kg to −11.24 kg with Compound 1 vs. −0.35 kg to −2.03 kg with placebo. During the dosing periods of multiple doses with Compound 1, decreases in mean body weight were observed in all treatment groups, at each time point assessed. The trend for decreased body weight appeared dose-dependent, with the mean changes from baseline in body weight observed at the end of the treatment period as follows:

| | |
|---|---|
| placebo: | −0.34 kg at Day 78 |
| Cohort 1: | −2.88 kg at Day 78 |
| Cohort 2: | −10.73 kg at Day 109. |

There were consistent statistically significant decreases in body weight from baseline, compared to placebo, in Cohort 2 starting at Day 50 and continued through Day 162 (i.e., 54 days after the last dose of study drug).

### Weight Circumference

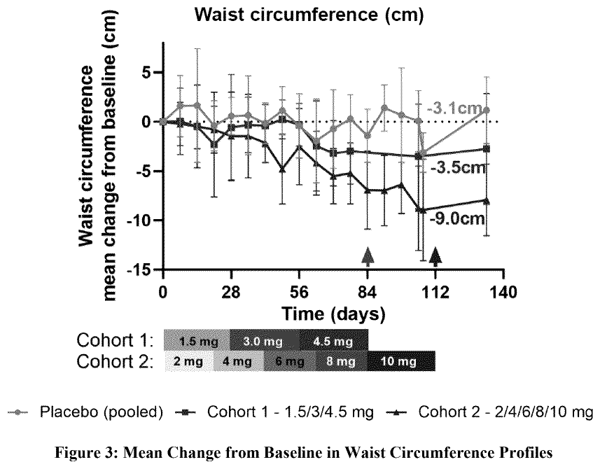

The waist circumference mean change from baseline is shown in FIG. 3. During the dosing periods of multiple doses with Compound 1, decreases in mean waist circumference were observed in both Cohort 1 and Cohort 2. Decreases in fat mass and increases in lean body mass were also observed in both Cohort 1 and Cohort 2 as shown in Table 3.

| | Cohort 1 | | | | | Placebo |
|---|---|---|---|---|---|---|
| | Baseline | Cohort 1 | Cohort 2 | Cohort 2 | Placebo | D78-D80 or |
| | D-2 | D78-80 | Baseline | D106-108 | Baseline | D106-108 |
| Waist circumference (cm) | 110.4 | 105.7 | 108.6 | 96.7 | 111.6 | 109.2 |
| Fat mass (kg) | 59.0 | 59.4 | 60.1 | 56.4 | 60.6 | 61.9 |
| Lean body Mass (kg) | 59.0 | 59.4 | 60.1 | 56.4 | 60.6 | 61.9 |
| % Lean body mass | 60.9% | 64.3% | 63.0% | 67.8% | 65.0% | 67.1% |

### Pharmacokinetics

A total of 355 Compound 1 PK concentrations from 18 patients are utilized for population PK modeling. All available PK data is utilized for model development. Table 4a lists Compound 1 final PK base model parameter estimates and corresponding between subject variabilities.

TABLE 4a
Compound 1 Population Pharmacokinetic Parameter Estimates

| | Population Mean | | Inter-individual |
|---|---|---|---|
| Parameters | Estimate (% SEE) | | Variability (% SEE) |
| Absorption rate constant, | 0.0299 | (12.2%) | 50.9 (43.9%) |
| KA (h⁻¹) | | | |
| Apparent volume of | 9.52 | (3.24%) | 11.0 (30.5%) |
| distribution, V/F (L) | | | |
| Apparent clearance, CL/F (L/h) | 0.0311 | (3.54%) | 14.4 (38.1%) |

Residual Error

| | | | |
|---|---|---|---|
| Proportional (%) | 0.0893 | (12.1%) | NA |
| Additive (ng/mL) | 8.89 | (33.1%) | NA |

Abbreviations: SEE = standard error of the estimate

Model-predicted mean (CV %) maximum steady state concentrations (C_{max,ss}), mean (CV %) steady state area under the concentration-time curves [AUC(0-168)ₛₛ] and estimated half-life (t_{1/2}) for Compound 1 at 4.5 mg QW and 10 mg QW are listed in Table 4b below.

TABLE 4b
Model-Predicted Compound 1 Steady-State
Pharmacokinetic Exposures and Half-life

| | Geometric Mean (CV %) | | | Geomean (Range) |
|---|---|---|---|---|
| | AUC(0-168)ₛₛ | C_{max, ss} | | t_{1/2} |
| Dose | (ng · h/mL) | (ng/mL) | | (h) |
| 4.5 mg QW | 106000 (22.5%) | 698 | (22.0%) | 209 (117 to 362) |
| Compound 1 | | | | |
| 10 mg QW | 239000 (23.9%) | 1570 | (23.1%) | 213 (129 to 366) |
| Compound 1 | | | | |

Abbreviations:
AUC(0-168)ₛₛ = steady state area under concentration-time curve over a weekly dosing interval;
C_{max, ss} = steady state maximum concentration;
CV % = coefficient of variation;
t_{1/2} = elimination half-life

### Glucose Control-HbA1c

The changes from baseline in HbA1c are shown in Table 4. During the 12 to 16 weeks of dosing, mean HbA1c levels decreased from baseline in all treatment groups, including placebo, from Day 29 (Week 5) onwards. Statistically significant decreases from baseline in HbA1c means, compared to placebo, were observed in both Cohort 1 and Cohort 2 treatment groups beginning on Day 57 and continued until Day 134.

Based on means, HbA1c levels remained decreased from baseline at Day 106 (i.e., 28 days after the last dose of study intervention Cohort 1) for all treatment groups.

TABLE 4
Mean Baseline and Change from Baseline in HbA1c (%)

| | | Change from Baseline | | | | | | |
|---|---|---|---|---|---|---|---|---|
| Treatment | | Day | Day | Day | Day | Day | Day | Day |
| Group | Baseline ^{a} | 29 | 43 | 57 | 64 | 78 | 85 | 106 |
| Cohort 1 | 8.52 | −0.70* | NC | −1.45* | NC | −1.80* | NC | −2.03* |
| Cohort 2 | 8.08 | NC | −0.83* | NC | −1.35* | NC | −1.50* | −1.67* |
| Placebo | 7.80 | −0.46 | −0.40 | −0.53 | −0.50 | −0.56* | −0.56* | −0.80* |

Abbreviations:
HbA1c = glycated haemoglobin; NC = not calculated.
^{a} Mean absolute value at Day − 2
*p-value < 0.05

### Glucose Control-Fasting Glucose

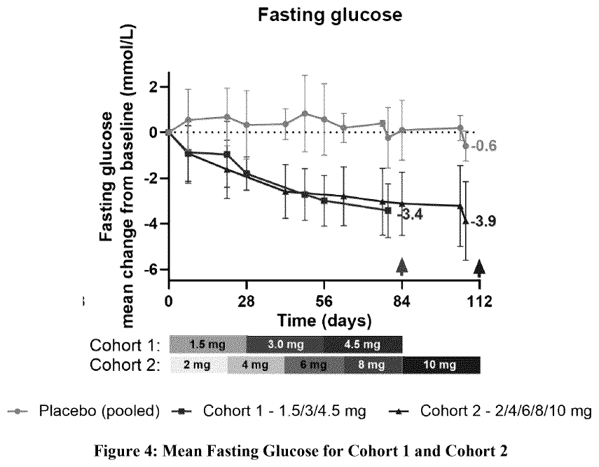

At baseline, the fasting glucose measurement were similar between treatment and placebo groups. Over the dosing period with multiple doses of Compound 1, mean fasting glucose levels decreased from baseline in all treatment groups as illustrated in FIG. 4. The greatest decreases from baseline at the end of the treatment period on Day 106 (Week 16) were observed in Cohort 2, when compared to placebo.

### Insulin Sensitivity-Fasting Insulin

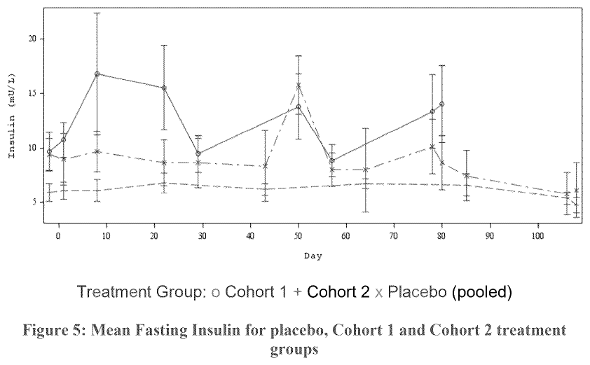

At baseline, the fasting insulin measurement is similar for the low dose cohort and placebo groups; however, the baseline fasting insulin levels for the higher dose cohort is much lower. FIG. 5 illustrates that increases in fasting insulin levels are observed in Cohort 1 whereas modest decreases in fasting insulin levels observed in Cohort 2, a pattern that is consistent with improved insulin sensitivity.

### Insulin Sensitivity-Fasting C-Peptide

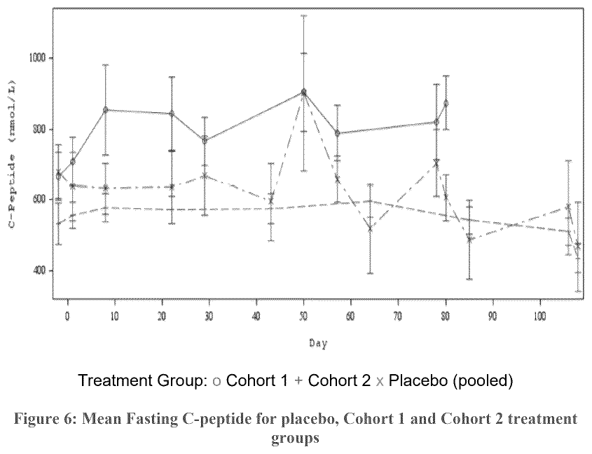

The changes from baseline in fasting c-peptide levels is shown in FIG. 6. At baseline, the fasting c-peptide measurements are similar between treatment and placebo groups. Over the dosing period with multiple doses of Compound 1, mean fasting c-peptide levels increased from baseline in all treatment groups, excluding the placebo group. However, mean fasting c-peptide levels decreased from baseline beginning on Day 106 and continued until Day 108 in Cohort 2, which is a pattern consistent with improved insulin sensitivity.

### Mixed Meal Tolerance Test

The changes from baseline in the AUC(0-3 h) of C-peptide, glucagon, glucose, insulin, oxyntomodulin during the MMTT are shown in Table 6.

TABLE 6
Mean Baseline and Change from Baseline in AUC(0-3
h) for the Parameters Assessed During the MMTT

| | | Change from |
|---|---|---|
| Mean Values | | Baseline |
| | Day 80 | Day 80 |
| Baseline^{a} | or 108^{b} | or 108^{b} |

C-peptide AUC(0-3 h) (pmol · h/L)

| | | | |
|---|---|---|---|
| Placebo | 5015.5 | 4806.7 | −50.9 |
| Cohort 1 | 4391.4 | 5850.1 | 1474.6* |
| Cohort 2 | 3604.1 | 3147.4 | −897.4 |

Glucagon AUC(0-3 h) (pmol · h/L)

| | | | |
|---|---|---|---|
| Placebo | 54.23 | 49.72 | −4.43 |
| Cohort 1 | 64.98 | 27.93 | −32.50 |
| Cohort 2 | 39.89 | 7.61 | −40.55 |

Glucose AUC(0-3 h) (mmol · h/L)

| | | | |
|---|---|---|---|
| Placebo | 38.58 | 33.78 | −5.54 |
| Cohort 1 | 43.06 | 25.72 | −14.37 |
| Cohort 2 | 38.27 | 23.74 | −15.73 |

Insulin AUC(0-3 h) (pmol · h/L)

| | | | |
|---|---|---|---|
| Placebo | 139.21 | 164.253 | 8.360 |
| Cohort 1 | 125.233 | 189.435 | 47.721 |
| Cohort 2 | 77.692 | 69.847 | −8.281 |

Oxyntomodulin AUC(0-3 h) (pmol · h/L)

| | | | |
|---|---|---|---|
| Placebo | 110.252 | 97.934 | −10.68 |
| Cohort 1 | 89.822 | 58.042 | −41.594 |
| Cohort 2 | 124.741 | 66.077 | −49.843 |

Abbreviations: AUC(0-3 h) = area under the concentration versus time curve from time zero to 3 hours postdose.
^{a}Mean absolute value at Day −2
^{b}Day 80 for Cohort 1 and Day 108 for Cohort 2

The changes from baseline in beta-cell function and insulin sensitivity parameters derived from the MMTT is shown in Table 7.

TABLE 7
Mean Baseline and Change from Baseline in Beta-Cell Function
and Insulin Sensitivity Parameters Derived from the MMTT

| Mean Values | | Change from Baseline | |
|---|---|---|---|
| | Day 80 | Day 80 | |
| Baseline^{a} | or 108^{b} | or 108^{b} | p-value |

Disposition index

| | | | | |
|---|---|---|---|---|
| Cohort 1 | 1.033 | 0.642 | −0.235 | 0.2837 |
| Cohort 2 | 1.276 | 1.265 | −0.198 | 0.917 |
| Placebo | 1.058 | 2.429 | 1.275 | 0.0154 |

HOMA2-B

| | | | | |
|---|---|---|---|---|
| Cohort 1 | 15.791 | 38.762 | 22.971 | <.0001 |
| Cohort 2 | 9.108 | 12.077 | 3.824 | 0.3977 |
| Placebo | 20.104 | 18.576 | −7.655 | 0.508 |

HOMA2-IR

| | | | | |
|---|---|---|---|---|
| Cohort 1 | 4.704 | 4.271 | −0.434 | 0.5257 |
| Cohort 2 | 2.7 | 1.325 | −1.071 | 0.396 |
| Placebo | 3.226 | 3.038 | −1.697 | 0.4433 |

ISI-M

| | | | | |
|---|---|---|---|---|
| Cohort 1 | 3.568 | 3.567 | 0.237 | 0.9026 |
| Cohort 2 | 4.616 | 10.7 | 5.905 | 0.0105 |
| Placebo | 2.747 | 3.891 | 0.936 | 0.6818 |

Second PH ISI-S

| | | | | |
|---|---|---|---|---|
| Cohort 1 | −6253.26 | −3634.241 | 2273.29 | <.0001 |
| Cohort 2 | −5889.19 | −3593.001 | 2252.292 | <.0001 |
| Placebo | −5717.28 | −4040.941 | 1266.106 | 0.0003 |

First PH ISI-S

| | | | | |
|---|---|---|---|---|
| Cohort 1 | −33534.1 | −19605.805 | 12090.422 | <.0001 |
| Cohort 2 | 31590.56 | −19372.347 | 11985.334 | <.0001 |
| Placebo | −30686.36 | −21765.086 | 6738.679 | 0.0003 |

Abbreviations:
HOMA2-B = Homeostatic Model Assessment of Beta Cell Function;
HOMA2-IR = Homeostatic Model Assessment of Insulin Resistance;
ISI-M = Matsuda Index;
First PH ISI-S = first phase Stumvoll Index;
Second PH ISI-S = second phase Stumvoll Index
^{a}Mean absolute value at Day −2
^{b}Day 80 for Cohort 1 and Day 108 for Cohort 2

### (i) Glucose AUC(0-3)h

Over the dosing period with multiple doses of Compound 1, the mean AUC(0-3 h) of glucose, as determined by MMTT, decreased from baseline in all treatment groups. The greatest decreases from baseline at the end of the treatment period on Day 108 (Week 16) are observed in Cohort 2, when compared to placebo.

### (ii) Insulin and C-Peptide AUC(0-3 h)

Over the 12-week dosing period with multiple doses of Compound 1, the mean AUC(0-3 h) of C-peptide and insulin, as determined by MMTT, is increased from mean baseline levels in the in Cohort 1. The greatest increases from baseline in mean AUC(0-3 h) for C-peptide at the end of the treatment period on Day 80 (Week 12) are observed in Cohort 1, when compared to placebo.

Over the 16-week dosing period with multiple doses of Compound 1, the mean AUC(0-3 h) of C-peptide and insulin, as determined by MMTT, is decreased from mean baseline levels in the in Cohort 2.

(iii) Beta-Cell Function

Over the dosing periods with multiple doses of Compound 1, mean HOMA2-B indices increased from baseline in both treatment groups. The greatest increase in mean HOMA2-B on Day 80 (Week 12) are observed in Cohort 1.

### (iv) Glucagon AUC(0-3h)

Over the dosing periods with multiple doses of Compound 1, the mean AUC(0-3 h) of glucagon, as determined by MMTT, decreased from baseline in all treatment groups. The greatest decreases from baseline at the end of the treatment period on Day 108 (Week 16) are observed in Cohort 2, when compared to placebo.

### (v) Insulin Sensitivity Parameters

Over the dosing periods with multiple doses of Compound 1, mean HOMA2-IR indices decreased and mean Matsuda insulin sensitivity index values increased from baseline in all treatment groups. In Cohort 1, glucose, insulin, and c-peptide excursions increased during the sMMTT resembling the pattern seen in GLP-1R mono-agonists and insulin secretagogues treatments.

### Lipids-Cholesterol, HDL, LDL and Triglycerides

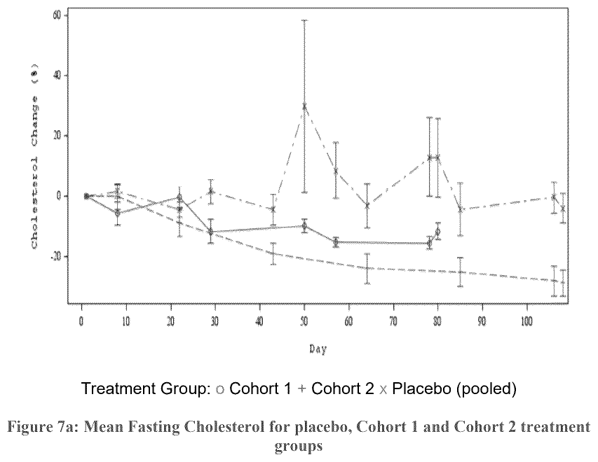

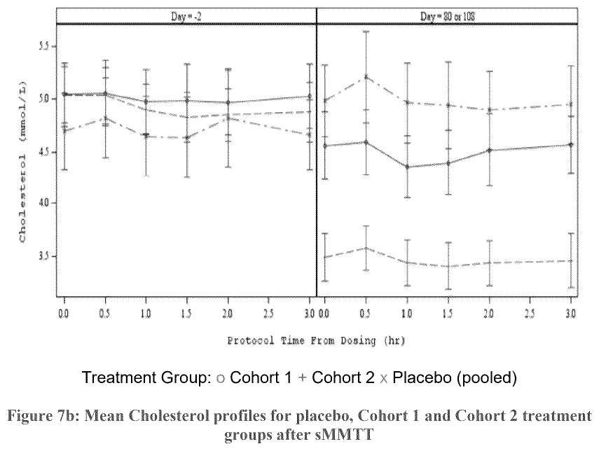

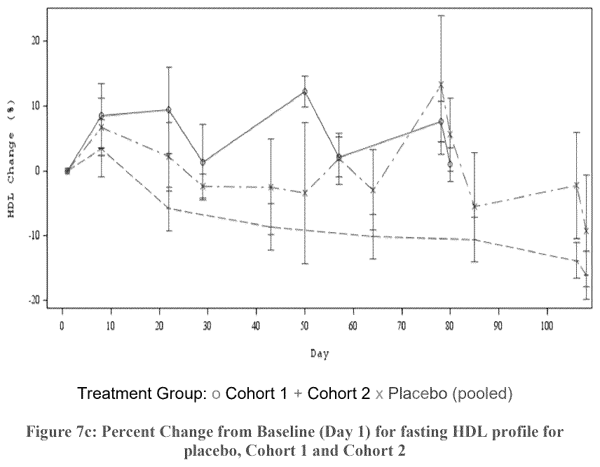

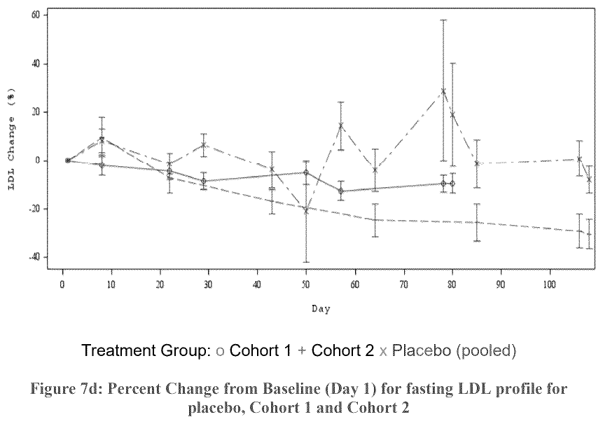

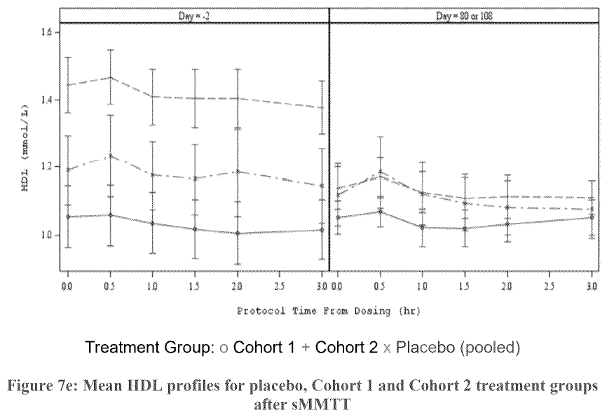

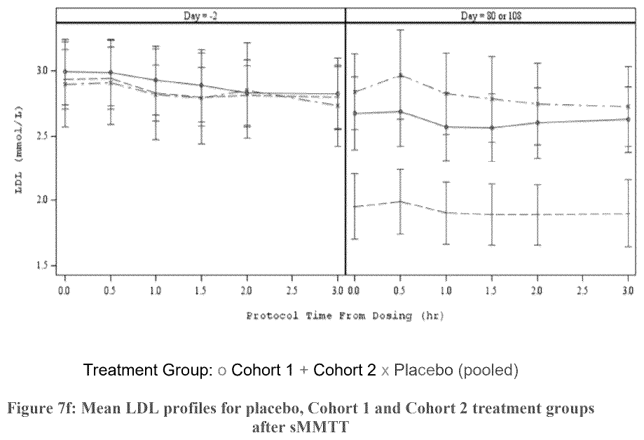

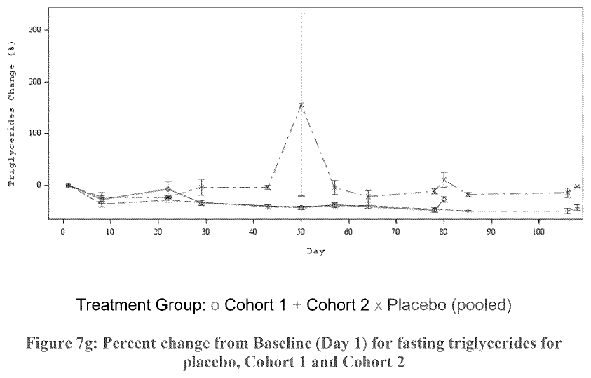

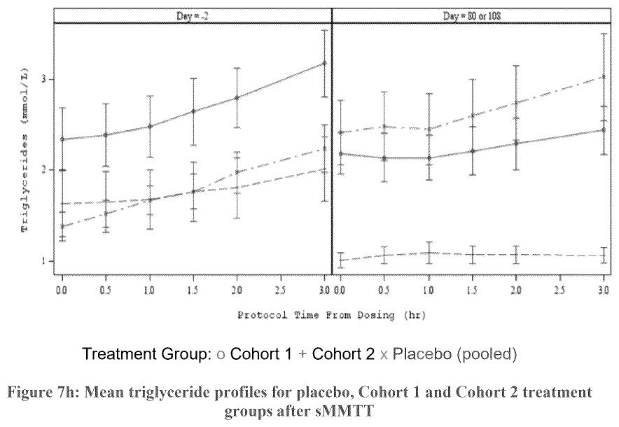

As shown in FIG. 7a, mean fasting cholesterol levels decreased from baseline in both treatment groups over the course of the dosing periods. Similarly, as shown in FIG. 7b, the Compound 1 Cohort 2 doses substantially reduced sMMTT total cholesterol levels.

As shown in FIGS. 7c and 7d, over the course of the dosing periods, mean fasting HDL and LDL levels decreased from baseline in Cohort 2. Similarly, as shown in FIGS. 7e and 7f, the Compound 1 Cohort 2 doses substantially reduce sMMTT LDL cholesterol levels and sMMTT HDL cholesterol levels.

As shown in FIG. 7g, over the course of the dosing periods, mean fasting triglyceride levels decreased from baseline in both treatment groups. Similarly, as shown in FIG. 7h, the Compound 1 Cohort 2 doses substantially reduce sMMTT triglyceride levels.

### Gastric Emptying

The PK parameters of acetaminophen were compared between treatment groups to determine the rate and extent of gastric emptying. The primary analysis of acetaminophen Cmax and Tmax are provided in Table 8. Prior to dosing with Compound 1, the PK profiles of acetaminophen on Day −1 were similar between the treatment groups. Within treatment groups, the Cmax of acetaminophen decreased from Day −1 during 12 weeks of multiple dosing with Compound 1. The Cmax of acetaminophen was lowest on Day 2 in both treatment groups. However, the change from baseline Cmax of acetaminophen increased at Day 107. The median tmax of acetaminophen was comparable between treatment groups and over time.

TABLE 8
Summary of Geometric Mean Cₘₐₓ and Median tₘₐₓ of Acetaminophen

| | | | | Change From | | | Change From | | | Change From | |
|---|---|---|---|---|---|---|---|---|---|---|---|
| | | | | Baseline to Day 2 | | Day | Baseline to Day 79 | | Day | Baseline to Day 107 | |
| | | Day − 1 | Day 2 | Mean | | 79 | Mean | | 107 | Mean | |
| | | Geo | Geo | (Est % | p- | Geo | (Est % | p- | Geo | (Est % | p- |
| | | Mean | Mean | change) | value | Mean | change) | value | Mean | change) | value |
| Cmax | Cohort 1 | 6617.56 | 5607.89 | −941.32 | 0.0302 | 6069.29 | −36.77 | 0.2024 | 8191.29 | 1487.25 | NC |
| | | | | (0.83) | | | (0.9) | | | (NC) | |
| | Cohort 2 | 6914.02 | 5513.14 | −1572.02 | 0.0072 | 7186.95 | −962.5 | NC | 8295.48 | 441.43 | 0.0685 |
| | | | | (0.79) | | | (NC) | | | (1.18) | |
| | Placebo | 8078.29 | 7699.28 | −460.02 | 0.9608 | NC | NC | 0.6893 | NC | NC | 0.7121 |
| | | | | (0.99) | | | (0.94) | | | (1.05) | |
| tmax, | Cohort 1 | 2.671 | 1.963 | −0.389 | 0.8445 | 1.478 | −0.907 | 0.4119 | 1.456 | 0.242 | NC |
| hr | | | | (1.06) | | | (0.8) | | | (NC) | |
| | Cohort 2 | 1.214 | 1.423 | 0.187 | 0.6685 | 3.302 | 0.333 | NC | 0.909 | 0.75 | 0.6851 |
| | | | | (0.89) | | | (NC) | | | (0.89) | |
| | Placebo | 1.31 | 1.091 | −0.333 | 0.2276 | NC | NC | 0.2425 | NC | NC | 0.4061 |
| | | | | (0.67) | | | (1.74) | | | (0.66) | |

Abbreviations:
Cₘₐₓ = maximum observed drug concentration; NC = not calculated; tₘₐₓ = time to maximum observed drug concentration.

### Appetite Analysis

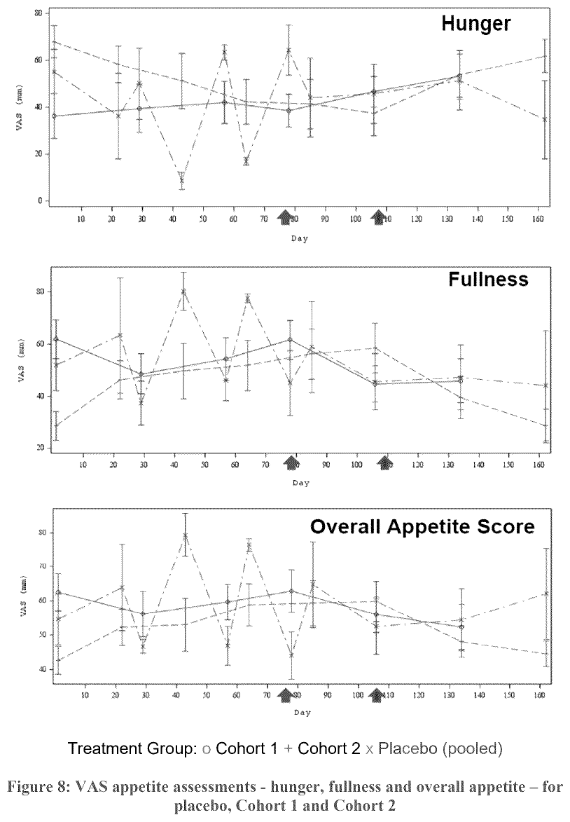

A summary of appetite sensations data across all time points is illustrated in FIG. 8. VAS appetite assessments showed reduced hunger, increased satiety, decreased overall appetite (higher overall score) in the higher dose cohort.

### (i) Overall Appetite Score

During multiple dosing with Compound 1, overall appetite scores generally increase from baseline in all treatment groups, including placebo, across the 12-week or 16-week dosing periods.

### (ii) Hunger

Over the dosing periods with multiple doses of Compound 1, hunger scores decrease from baseline at the majority of time points assessed in both treatment groups, including placebo.

(iii) Satiety

Over the dosing periods with multiple doses of Compound 1, satiety scores decrease from baseline in both treatment groups. The satiety scores of patients in the placebo group increased from baseline at Day 80.

### (iv) Fullness

Over the dosing periods with multiple doses of Compound 1, fullness scores increased from baseline in both treatment groups. The fullness scores of patients in the placebo group decreased from baseline at Day 80. The greatest increase from baseline in fullness scores are observed in Cohort 1.

### Pharmacodynamic Assessment of Differences as Doses are Escalated

### Fasting Glucose

Patients in Cohort 1 have a Mean Baseline Fasting Plasma Glucose of 10.78 mmol/L. As doses of Compound 1 are escalated to 4.5 mg, mean fasting glucose decrease to 6.52 mmol/L at Day 80, resulting in an approximate reduction of 40%.

Patients in Cohort 2 have a mean baseline fasting glucose of 10.46 mmol/L. As doses of Compound 1 are escalated to 10.0 mg, mean fasting glucose decreases to 5.91 mmol/L at Day 108, resulting in an approximate reduction of 43%.

### Fasting Insulin

Patients in Cohort 1 have a mean fasting baseline plasma insulin level of 9.648 mIU/L. As doses of Compound 1 are escalated to 4.5 mg, mean fasting insulin levels increased to 13.362 mIU/L at Day 78 and to 14.046 mIU/L at Day 80, resulting in an approximate increase from baseline of 38-46%.

Patients in Cohort 2 have a mean fasting baseline insulin level of 5.892 mIU/L. As doses of Compound 1 are escalated to 6.0 mg, mean fasting insulin levels increase to 6.73 mIU/L, resulting in an approximate increase from baseline of 14%. As doses of Compound 1 are escalated to 8.0 mg, mean fasting insulin levels are 6.589 mIU/L, an approximate increase from baseline of 12%. As doses of Compound 1 are escalated to 10.0 mg, mean fasting insulin levels decrease to 5.355 mIU/L at Day 106 and to 4.74 mIU/L at Day 108, resulting in an approximate decrease of fasting insulin from baseline of 20%. This data indicates that with dose of Compound 1 to greater than 8.0 mg, fasting plasma glucose can be reduced without increasing fasting insulin levels, consistent with reduced insulin resistance.

### C-Peptide

Patients in Cohort 1 have a mean fasting baseline plasma C-peptide level of 667 pmol/L. As doses of Compound 1 are escalated to 4.5 mg, mean fasting C-peptide levels increase to 820.2 μmol/L at Day 78 and to 874.9 μmol/L at Day 80, resulting in an approximate increase from baseline of 22-31%.

Patients in Cohort 2 have a mean fasting baseline C-peptide level of 532.8 μmol/L. As doses of Compound 1 are escalated to 6.0 mg, mean fasting C-peptide level increases to 596.6 μmol/L, resulting in an approximate increase from baseline of 12%. As doses of Compound 1 are escalated to 8.0 mg, the mean fasting C-peptide level increases only slightly from baseline to 541.7 μmol/L (approximately 2%). As doses of Compound 1 are escalated to 10.0 mg, mean fasting C-peptide levels decreased to 511.1 μmol/L at Day 106 and to 435.4 μmol/L at Day 108, resulting in an approximate decrease of fasting C-peptide level from baseline of 18%. This data indicates that with dose of Compound 1 to greater than 8.0 mg, fasting plasma glucose can be reduced without increasing fasting C-peptide secretion from the pancreatic beta cell, consistent with reducing pancreatic beta-cell workload.

### Fasting Glucagon

Patients in Cohort 1 have a mean fasting baseline plasma glucagon level of 14.79 pmol/L. As doses of Compound 1 are escalated to 4.5 mg, mean fasting glucagon level decreases to 6.65 μmol/L at Day 78 and to 8.19 μmol/L at Day 80, resulting in an approximate decrease from baseline of 45-55%.

Patients in Cohort 2 have a mean fasting baseline glucagon level of 9.83 μmol/L. As doses of Compound 1 are escalated to 6.0 mg, mean fasting glucagon level decreases to 3.65 μmol/L, resulting in an approximate decrease from baseline of 63%. As doses of Compound 1 are escalated to 8.0 mg, the mean fasting glucagon level decreases from baseline at Day 80 to 2 μmol/L. As doses of Compound 1 are escalated to 10.0 mg, mean fasting glucagon level decreased from baseline at Day 106 to 1.98 μmol/L and at Day 108 to 1.93 μmol/L. This data indicates that with dose of Compound 1 to greater than 6.0 mg, fasting plasma glucagon can be reduced by approximately 80%.

### Fasting Total Cholesterol

Patients in Cohort 1 have a mean fasting total cholesterol level of 5.065 mmol/L. As doses of Compound 1 are escalated to 4.5 mg, mean fasting total cholesterol levels decrease to 4.275 mmol/L at Day 78 and to 4.461 mmol/L at Day 80, resulting in an approximate decrease from baseline of 12-16%.

Patients in Cohort 2 have a mean fasting baseline total cholesterol level of 4.971 mmol/L. As doses of Compound 1 are escalated to 6.0 mg, mean fasting total cholesterol level decreases at Day 64 to 3.673 mmol/L, resulting in an approximate decrease from baseline of 26%. As doses of Compound 1 are escalated to 8.0 mg, the mean fasting total cholesterol level decreases at Day 85 from baseline to 3.614 mmol/L (approximately 27%). As doses of Compound 1 are escalated to 10.0 mg, mean total cholesterol levels decrease to 3.454 mmol/L at Day 106 and to 3.441 mmol/L at Day 108, resulting in an approximate decrease of fasting total cholesterol levels from baseline of 31%. This data indicates that with dose of Compound 1 to greater than 8.0 mg, fasting total cholesterol levels can be reduced by more than 30%.

### Fasting LDL Cholesterol

Patients in Cohort 1 have a mean fasting LDL cholesterol level of 2.852 mmol/L. As doses of Compound 1 are escalated to 4.5 mg, mean fasting LDL cholesterol levels decrease to 2.56 mmol/L at Day 78 and at Day 80, resulting in an approximate decrease from baseline of 10%.

Patients in Cohort 2 have a mean fasting baseline LDL cholesterol level of 2.796 mmol/L. As doses of Compound 1 are escalated to 6.0 mg, mean fasting LDL cholesterol level decrease at Day 64 to 1.997 mmol/L, resulting in an approximate decrease from baseline of 29%. As doses of Compound 1 are escalated to 8.0 mg, the mean fasting LDL cholesterol level decreases from baseline at Day 85 to 1.954 mmol/L (approximately 30%). As doses of Compound 1 are escalated to 10.0 mg, mean fasting LDL cholesterol levels decrease to 1.859 mmol/L at Day 106 and to 1.849 mmol/L at Day 108, resulting in an approximate decrease of fasting LDL cholesterol levels from baseline of 33-34%. This data indicates that with dose of Compound 1 to 8.0 mg or higher, fasting LDL cholesterol levels can be reduced by 30% or greater.

### Fasting Triglyceride

Patients in Cohort 1 have a mean fasting triglyceride level of 2.938 mmol/L. As doses of Compound 1 are escalated to 4.5 mg, mean fasting triglyceride levels decreases to 1.456 mmol/L at Day 78 and to 2.084 mmol/L at Day 80, resulting in an approximate decrease from baseline of 29-50%.

Patients in Cohort 2 have a mean fasting baseline triglyceride level of 1.867 mmol/L. As doses of Compound 1 are escalated to 6.0 mg, mean fasting triglyceride level decrease at Day 64 to 1.055 mmol/L, resulting in an approximate decrease from baseline of 43%. As doses of Compound 1 are escalated to 8.0 mg, the mean fasting triglyceride level decrease from baseline at Day 85 to 0.855 mmol/L (approximately 54%). As doses of Compound 1 are escalated to 10.0 mg, mean fasting triglyceride 1 levels decrease to 0.86 mmol/L at Day 106 and to 0.985 mmol/L at Day 108, resulting in an approximate decrease of fasting triglyceride levels from baseline of 47-54%. This data indicates that with dose of Compound 1 to greater than 6.0 mg, fasting triglyceride levels can be reduced by 50% or greater.

### Body Weight

Patients in Cohort 1 have a mean baseline body weight of 97.19 kg in which doses of LY were escalated to 4.5 mg, mean body weight decreased at Day 78 to 94.0 kg with a mean percent change from baseline of −3.03%.

Patients in Cohort 2 have a mean baseline body weight of 94.9 kg. As doses of Compound 1 are escalated to 4.0 mg, mean body weight decreases at Day 43 to 90.38 kg, with a mean percent change from baseline of −4.04%. As doses of Compound 1 are escalated to 6.0 mg, mean body weight decreases at Day 64 to 87.69 kg, with a mean percent change from baseline of −7.07%. As doses of Compound 1 are escalated to 8.0 mg, mean body weight decreases at Day 85 to 85.88 kg, with a mean percent change from baseline of −9.05%. As doses of Compound 1 are escalated to 10 mg, mean body weight decreased at Day 109 to 82.55 kg, with a mean percent change from baseline of −12.71%.

Body weight remained decreased from baseline after the conclusion of 16 weeks of Compound 1 dose and administration. At Day 134, mean body weight of the Compound 1 arm in Cohort 2 is 84.05 kg, with a mean percent change from baseline of −11.14%. At Day 162, mean body weight of the Compound 1 arm in the second cohort is 88.96 kg, with a mean percent change from baseline of −6.56%. These results indicate that dose of Compound 1 to greater than 6 mg can result in body weight reductions of greater than 9%. Furthermore, dose of Compound 1 to 10 mg or greater can reduce body weight by greater than 12%.

### Waist Circumference

Patients in Cohort 1 have a mean baseline waist circumference of 108.66 cm. in which doses of LY were escalated to 4.5 mg, mean waist circumference decreased at Day 78 to 105.66 cm, approximately a 3% reduction from baseline.

Patients in Cohort 2 have a mean baseline waist circumference of 106.93 cm. As doses of Compound 1 are escalated to 4.0 mg, mean waist circumference decreases at Day 43 to 103.38 cm, approximately a 3% reduction from baseline. As doses of Compound 1 are escalated to 6.0 mg, mean waist circumference decreases at Day 64 to 101.31 cm, approximately a 5% reduction from baseline. As doses of Compound 1 are escalated to 8.0 mg, mean waist circumference decreased at Day 85 to 98.58 cm, approximately an 8% reduction from baseline. As doses of Compound 1 are escalated to 10 mg, mean waist circumference decreased at Day 109 to 96.56 cm, approximately a 10% reduction from baseline.

Waist circumference remained decreased from baseline after the conclusion of 16 weeks of LY dose and administration. At Day 134, mean waist circumference of the Compound 1 arm in Cohort 2 was 97.59 cm, approximately a 9% reduction from baseline. At Day 162, mean waist circumference of the Compound 1 arm in the second cohort is 102.79 cm, approximately a 4% reduction from baseline. This data indicates that Compound 1 dose greater than 6.0 mg can result in improvements in body composition with reductions in waist circumference by greater than 5%.

### Fasting Insulin Resistance

Patients in Cohort 1 have a mean fasting insulin resistance HOMA-IR index of 4.704. As doses of Compound 1 are escalated to 4.5 mg, mean HOMA-IR index decreases to 4.289 at Day 78 and to 4.271 at Day 80, resulting in an approximate decrease from baseline of 9%.

Patients in Cohort 2 have a mean fasting insulin resistance HOMA-IR index of 2.7. As doses of Compound 1 are escalated to 6.0 mg, mean HOMA-IR index decreased at Day 64 to 2.158, resulting in an approximate decrease from baseline of 20%. As doses of Compound 1 are escalated to 8.0 mg, the mean HOMA-IR index decreased from baseline at Day 85 to 1.976 (approximately 27%). As doses of Compound 1 are escalated to 10.0 mg, mean HOMA-IR index decreased to 1.325 at Day 108, resulting in an approximate decrease of HOMA-IR from baseline of 51%. This data indicates that with dose of Compound 1 to greater than 6.0 mg, fasting insulin resistance indices can be reduced by greater than 25%. With dose of Compound 1 to 10 mg or greater, fasting insulin resistance indices can be reduced by 50%.

### Fasting Pancreatic Beta-Cell

Patients in Cohort 1 have a mean fasting pancreatic beta-cell HOMA-B index of insulin secretion of 15.791. As doses of Compound 1 are escalated to 4.5 mg, mean HOMA-B index increased to 38.762 at Day 80, resulting in an approximate increase from baseline of 145%.

Patients in Cohort 2 have a mean fasting pancreatic beta-cell HOMA-B index of insulin secretion of 9.108. As doses of Compound 1 are escalated to 6.0 mg, mean HOMA-B index was increased from baseline at Day 64 to 16.072, resulting in an approximate increase of 76%. As doses of Compound 1 are escalated to 8.0 mg, the mean HOMA-B index was increased from baseline at Day 85 to 16.689 (approximately 83%). As doses of Compound 1 are escalated to 10.0 mg, mean HOMA-B index increased from baseline at Day 108 to 12.077, resulting in an approximate increase of HOMA-B from baseline of 33%. This data indicates that with dose of Compound 1 to greater than 8.0 mg, increases in fasting HOMA-B indices of insulin secretion are attenuated, consistent with improvements in fasting insulin sensitivity and reduced requirements for insulin secretion in the setting of reduced fasting glucose levels.

### MMTT—Glucose Levels

Patients in Cohort 1 have a baseline mean area under the curve 0-3 hours for glucose levels during mixed meal tolerance testing was 43.06 h*mmol/L. As doses of Compound 1 are escalated to mg, mean area under the curve 0-3 hours for glucose levels decreased to 25.76 h*mmol/L at Day 80, resulting in an approximate decrease from baseline of 40%.

Patients in Cohort 2 have a baseline mean area under the curve 0-3 hours for glucose levels during mixed meal tolerance testing of 38.27 h*mmol/L. As doses of Compound 1 are escalated to 10.0 mg, area under the curve 0-3 hours for glucose levels decreased to 25.76 h*mmol/L at Day 108, resulting in an approximate decrease of meal-stimulated glucose levels from baseline of 38%.

### MMTT—Insulin Levels

Patients in Cohort 1 have a baseline mean area under the curve 0-3 hours for insulin levels during mixed meal tolerance testing was 125.233 h*mIU/L. As doses of Compound 1 are escalated to 4.5 mg, mean area under the curve 0-3 hours for insulin levels increased to 189.435 h*mIU/L at Day 80, resulting in an approximate increase from baseline of 51%.

Patients in Cohort 2 have a baseline mean area under the curve 0-3 hours for insulin levels during mixed meal tolerance testing of 77.692 h*mIU/L. As doses of Compound 1 are escalated to 10.0 mg, area under the curve 0-3 hours for insulin levels decreased to 69.847 h*mIU/L at Day 108, resulting in an approximate decrease of meal-stimulated insulin levels from baseline of 10%. This data indicate that dose of Compound 1 up to 4.5 mg stimulates insulin secretion from baseline in response to mixed meals. Dose of Compound 1 greater than 4.5 mg and up to 10 mg does not stimulate insulin secretion from baseline in response to mixed meals, consistent with reducing pancreatic beta cell workload while also lowering glucose levels.

### MMTT—C-Peptide

Patients in Cohort 1 have a baseline mean area under the curve 0-3 hours for C-peptide levels during mixed meal tolerance testing was 4391.4 h*pmol/L. As doses of Compound 1 are escalated to 4.5 mg, mean area under the curve 0-3 hours for C-peptide levels increased to 5850.1 h*pmol/L at Day 80, resulting in an approximate increase from baseline of 33%.

Patients in Cohort 2 have a baseline mean area under the curve 0-3 hours for C-peptide levels during mixed meal tolerance testing of 3604.1 h*pmol/L. As doses of Compound 1 are escalated to 10.0 mg, area under the curve 0-3 hours for C-peptide levels decreased to 3147.4 h*pmol/L at Day 108, resulting in an approximate decrease of meal-stimulated C-peptide levels from baseline of 13%. This data indicates that dose of Compound 1 up to 4.5 mg stimulate insulin secretion with proinsulin processing from baseline in response to mixed meals. Dose of Compound 1 greater than 4.5 mg and up to 10 mg does not stimulate insulin secretion with proinsulin processing from baseline in response to mixed meals, consistent with reducing pancreatic beta-cell workload while also lowering glucose levels.

### MMTT—Glucagon

Patients in Cohort 1 have a baseline mean area under the curve 0-3 hours for glucagon levels during mixed meal tolerance testing of 64.98 h*pmol/L. As doses of Compound 1 are escalated to 4.5 mg, mean area under the curve 0-3 hours for glucagon levels decreased to 27.93 h*pmol/L at Day 80, resulting in an approximate decrease from baseline of 57%.

Patients in Cohort 2 have a baseline mean area under the curve 0-3 hours for glucagon levels during mixed meal tolerance testing of 39.89 h*pmol/L. As doses of Compound 1 are escalated to 10.0 mg, area under the curve 0-3 hours for glucagon levels decreased to 7.61 h*pmol/L at Day 108, resulting in an approximate decrease of meal-stimulated glucagon levels from baseline of 81%. This data indicates that dose of Compound 1 up to 4.5 mg reduces glucagon secretion from baseline by greater than 50% in response to mixed meals. Dose of Compound 1 up to 10 mg reduces glucagon secretion by greater than 80% from baseline in response to mixed meals.

### MMTT—Matsuda Insulin Sensitivity Index (ISI-M)

Patients in Cohort 1 have a baseline Matsuda insulin sensitivity index (ISI-M) calculated from mixed meal tolerance test parameters of 3.568. As doses of Compound 1 are escalated to 4.5 mg, mean Matsuda index was 3.567 at Day 80, indicative of no change from baseline.

Patients in Cohort 2 have a baseline Matsuda insulin sensitivity index (ISI-M) calculated from mixed meal tolerance test parameters of 4.616. As doses of Compound 1 are escalated to 10.0 mg, Matsuda index increased to 10.7 at Day 108, an increase from baseline of approximately 132% that was indicative of increased insulin sensitivity. This data indicates that dose of Compound 1 up to 4.5 mg does not improve insulin sensitivity in response to mixed meals. Dose of Compound 1 up to 10 mg notably increases insulin sensitivity from baseline in response to mixed meals.

### Adverse Events

No deaths occurred during this study. Throughout the study, a total of 17 (94.4%) of patients that received Compound 1 reported TEAEs and 6 (100%) of patients that received placebo reported TEAEs as summarized in Table 9. The majority of TEAEs reported during the study were mild (88.5%) in severity. Overall, the most frequently reported TEAEs by participants that received LY3305677 are gastrointestinal disorders:
nausea 7 (38.9%),
vomiting 5 (27.8%),
eructation 4 (22.2%),
diarrhea 4 (22.2%),
abdominal pain 1 (5.6%), and
constipation 2 (11.1%).

TABLE 9
Summary of Treatment-Emergent Adverse Events (All Causalities)

| | | | | Number of adverse events | | |
|---|---|---|---|---|---|---|
| | Number of | | | [number of patients with adverse events] | | |
| | patients | | | (percentage of patients with adverse events) | | |
| Treatment | [%] with | | | | | Leading to |
| arm | adverse events | | Severity | Overall | Serious | discontinuation |
| Cohort 1 | 8 | [88.9] | Mild | 33 [8] (88.9) | 0 [0] (0.0) | 0 [0] (0.0) |
| (N = 9) | | | Moderate | 2 [1] (11.1) | 1 [1] (2.2) | 0 [0] (0.0) |
| | | | Severe | 0 [0] (0.0) | 0 [0] (0.0) | 0 [0] (0.0) |
| | | | Total | 35 [8] (88.9) | 1 [1] (2.2) | 0 [0] (0.0) |
| Cohort 2 | 9 | [100] | Mild | 79 [9] (100) | 0 [0] (0.0) | 0 [0] (0.0) |
| (N = 9) | | | Moderate | 12 [4] (44.4) | 1 [1] (11.1) | 1 [1] (11.1) |
| | | | Severe | 0 [0] (0.0) | 0 [0] (0.0) | 0 [0] (0.0) |
| | | | Total | 91 [9] (100) | 1 [1] (11.1) | 1 [1] (11.1) |
| Total | 17 | [94.4] | Mild | 112 [17] (94.4) | 0 [0] (0.0) | 0 [0] (0.0) |
| Compound 1 | | | Moderate | 14 [5] (27.8) | 0 [0] (0.0) | 0 [0] (0.0) |
| (N = 18) | | | Severe | 0 [0] (0.0) | 0 [0] (0.0) | 0 [0] (0.0) |
| | | | Total | 1 [17] (94.4) | 0 [0] (0.0) | 0 [0] (0.0) |
| Placebo | 6 | [100] | Mild | 11 [6] (100) | 0 [0] (0.0) | 0 [0] (0.0) |
| (N = 6) | | | Moderate | 2 [2] (33.3) | 1 [1] (3.6) | 0 [0] (0.0) |
| | | | Severe | 0 [0] (0.0) | 1 [1] (3.6) | 0 [0] (0.0) |
| | | | Total | 13 [6] (100) | 2 [2] (7.1) | 0 [0] (0.0) |
| Overall | 23 | [95.8] | Mild | 123 [23] (95.8) | 0 [0] (0.0) | 0 [0] (0.0) |
| (N = 24) | | | Moderate | 16 [7] (29.2) | 1 [1] (4.17) | 1 [1] (4.17) |
| | | | Severe | 0 [0] (0.0) | 0 [0] (0.0) | 0 [0] (0.0) |
| | | | Total | 139[23] (95.8) | 1 [1] (4.17) | 1 [1] (4.17) |

Abbreviations: N = number of patients

During this study, one SAE was reported in 1 patient treated with study intervention as shown in Table 10.

TABLE 10
SAEs Reported

| Last | | | | | Action |
|---|---|---|---|---|---|
| treatment | Study | MedDRA | | Event | taken with |
| prior to | Day at | preferred | | rela- | study |
| SAE | Onset^{a} | term | Severity | tionship | treatment |
| Compound | 110 | Sarcoidosis | Moderate | Unrelated | Drug |
| 1 | | | | to study | withdrawn |
| | | | | treatment | |

Abbreviations: MedDRA = Medical Dictionary for Regulatory Activities; SAE = serious adverse event a Derived based on the first dose of study drug administered on Day 1.

### Example 2: Clinical Study

### Overview

This study is designed to evaluate the safety, tolerability, PD and PK of dose to higher doses of Compound 1 in individuals with obesity. The Multiple Ascending Dose (MAD) study is a randomized, investigator- and participant-blind, placebo-controlled, multiple-ascending dose, single- or multi-site study conducted in individuals with obesity (i.e., females or males aged 18-70 years, inclusive, a BMI in the range of 27 kg/m² to 50 kg/m²). Participants should have stable body weight for at least 3 months prior to the initiation of the study.

### Treatment

Dose of Compound 1 is assessed with weekly dosing over 20 weeks in 2 cohorts. Each cohort comprises 12 patients-nine patients are administered Compound 1 and three patients are administered placebo.

The dose for Cohort 1 is as follows:
1.5 mg Compound 1 for 2 weeks;
3.0 mg Compound 1 for 2 weeks;
6.0 mg Compound 1 for 4 weeks;
8.0 mg Compound 1 for 4 weeks;
12.0 mg Compound 1 for 4 weeks; and
16.0 mg Compound 1 for 4 weeks.

The dose for Cohort 2 is as follows:
2.0 mg Compound 1 for 3 weeks;
4.0 mg Compound 1 for 3 weeks;
6.0 mg Compound 1 for 3 weeks;
8.0 mg Compound 1 for 3 weeks;
10.0 mg Compound 1 for 2 weeks;
13.0 mg Compound 1 for 2 weeks, and
16.0 mg Compound 1 for 4 weeks.

### Assessments

Blood samples are periodically collected from Day 1 through Day 138 to determine plasma concentrations across increasing doses of Compound 1 in individuals with obesity.

HbA1c, C-peptide, glucagon, serum glucose and insulin are measured at fasting at multiple time points across dose from Day 1 through Day 138 and for a mixed meal tolerance test (MMTT) at baseline before initial dose and during Week 20 using methods known in the art for each of the analytes. Mean absolute and percent change in fasting glucose, insulin, C-peptide, and glucagon are calculated for Compound 1 and for placebo arms within each cohort and compared to baseline at multiple time points from Day 1 through Day 138.

Parameters derived from fasting samples and from the MMTT to assess insulin resistance and pancreatic β cell function will include the following:
Homeostatic model assessment (HOMA)-B;
HOMA-IR; and
insulin sensitivity (Matsuda Index).

Insulin sensitivity (Matsuda Index) is calculated as per the below equation:

$$ISI = \frac{10000}{\sqrt{G 0 * I 0 * \frac{GlucoseAUC ( 0 - 2 h )}{2 h} * \frac{InsulinAUC ( 0 - 2 h )}{2 h}}} ,$$

where glucose is in mg/dL and insulin is in μU/mL.

Mean parameters for multiple indices related to glycemic control, pancreatic beta-cell function, and insulin sensitivity at Day 138 will be derived from fasting and MMTT measures for Compound 1 and Placebo arms within each cohort and compared to baseline.

Satiety VAS is measured at fasting for multiple time points across dose from Day 1 through Day 138. Appetite sensations are assessed using a VAS for parameters of hunger, fullness, satiety and prospective food intake, and an overall appetite score is calculated.

Body weight and waist circumference are measured at frequent intervals across dose from Day 1 through Day 138 and for an extended follow-up period after the conclusion of dosing. Mean absolute and percent change in body weight are calculated for Compound 1 and for placebo arms within each cohort and compared to baseline at multiple time points from Day 1 through Day 138 and throughout the extended follow-up period after the conclusion of dosing. Mean absolute and percent change in waist circumference are calculated for Compound 1 and for placebo arms within each cohort and compared to baseline at multiple time points from Day 1 through Day 138 and throughout the extended follow-up period after the conclusion of dosing. While the weight loss described in Example 1 is in a T2D patient population, greater weight loss is typical in an obesity patient population. A multiplier of 1.6-1.8 may be applied to percent weight loss moving from the T2D population to an obesity population.

Lipid profiles, including total cholesterol, LDL cholesterol, VLDL cholesterol, HDL cholesterol, and triglycerides are measured at fasting for multiple time points across dose from Day 1 through Day 138. Mean absolute and percent change in fasting lipid profiles, including total cholesterol, LDL cholesterol, VLDL cholesterol, HDL cholesterol, and triglycerides are calculated for Compound 1 and for Placebo arms within each cohort and compared to baseline at multiple time points from Day 1 through Day 138.

Vital signs are monitored at frequent intervals across dose from Day 1 through Day 138, including heart rate, pulse rate, systolic blood pressure, and diastolic blood pressure.

Safety parameters assessed during the study include: AEs (with special interest to GI AEs such as nausea, vomiting, and diarrhea), laboratory parameters, physical examinations/medical assessments, vital signs and ECGs, BW, injection site reactions, hypersensitivity reactions, and glucose monitoring (with special attention to hypoglycemia).

### Body Weight

During the dosing periods of multiple doses with Compound 1, decreases in mean body weight were observed in all treatment groups, at each time point assessed. The trend for decreased body weight was dose-dependent, with the mean percent changes from baseline in body weight observed at the end of the treatment period as follows:

| | |
|---|---|
| placebo: | +0.5% at Day 138 (week 20) |
| Cohort 1: | −19.4% at Day 138 (week 20) |
| Cohort 2: | −20.6% at Day 138 (week 20) |

The weight loss data at various time points in the two cohorts is as follows:
Day 85/12 weeks (after 4 weeks of 8 mg): placebo +0.2%, Cohort 1-10.7%, Cohort 2 −11.7%
Day 99/14 weeks (Cohort 2 after 2 weeks of 10 mg): placebo +0.07%, Cohort 2 −13.4%
Day 113/16 weeks (Cohort 1 after 4 weeks of 12 mg; Cohort 2 after 2 weeks of 13 mg): placebo +0.7%, Cohort 1 −15.4%, Cohort 2 −16.2%
Day 138/20 weeks (after 4 weeks of 16 mg): placebo +0.5%, Cohort 1 −19.4%, Cohort 2 −20.6%.

### Waist Circumference

During the dosing periods of multiple doses with Compound 1, decreases in mean waist circumference were observed in both Cohort 1 and Cohort 2.
Day 85/12 weeks (after 4 weeks of 8 mg) placebo +0.04%, Cohort 1 −5.2%, Cohort 2 −7.6%
Day 99/14 weeks (Cohort 2 after 2 weeks of 10 mg) placebo +1.0%, Cohort 2 −6.8%
Day 113/16 weeks (Cohort 1 after 4 weeks of 12 mg; Cohort 2 after 2 weeks of 13 mg) placebo +1.3%, Cohort 1 −6.3%, Cohort 2 −12.6%
Day 138/20 weeks (after 4 weeks of 16 mg) placebo +0.4%, Cohort 1 −11.5%, Cohort 2 −16.6%

### Adverse Events

No deaths occurred during this study. No serious adverse events were observed. The most common treatment-emergent adverse events with mazdutide were gastrointestinal in nature (nausea, vomiting, diarrhea), consistent with GLP-1 receptor agonists. The incidences of nausea, vomiting, diarrhea were similar across the high doses of 10-16 mg, and were mild or moderate in severity.

### Example 3: Clinical Study

### Overview

This study is designed to further evaluate the effectiveness and safety of the Compound 1 in a population of obese individuals or overweight individuals. The study is a randomized, investigator- and participant-blind, placebo-controlled study conducted in obese individuals or overweight individuals without diabetes (i.e., females or males aged 18-75 years, inclusive, with obesity prior to randomization and with a BMI of ≥30 kg/m² at screening or overweight prior to randomization and with a BMI of >27 kg/m² or <30 kg/m² at screening with one or more weight-related comorbidities, such as hypertension, dyslipidemia, cardiovascular disease, osteoarthritis or obstructive sleep apnea, but without T2DM). Participants should have stable body weight for at least 3 months prior to the initiation of the study.

### Treatment

Multiple doses of Compound 1 are assessed over 48 weeks in cohorts administered the Compound 1, or placebo. Approximately 60 individuals are included in each cohort.

Multiple cohorts are administered Compound 1 from Week 1 up to Week 20 or Week 32 to achieve distinct maintenance or highest doses administered between Week 21 or Week 32 (or the earliest week that dose is first achieved) through Week 48. Starting doses of dose regimens may vary by cohort, for example with starting doses of 1.5 mg, 2.0 mg, or 3.0 mg. Time intervals of dose steps may vary within and across cohorts, including dose increases after 2 week, 3 week, or 4 week intervals. Magnitude of dose increases may vary within and across cohorts, with dose increases of 1.5 mg, 2.0 mg, 3.0 mg, 4.0 mg, or 5.0 mg occurring across dose steps to achieve a designated dose.

In this study, the first dosing regimen (Cohort 1) is as follows:
1.5 mg QW Compound 1 for 4 weeks;
3.0 mg QW Compound 1 for 28 weeks; and
6 mg QW Compound 1 for 16 weeks.

The second dosing regimen (Cohort 2) is as follows:
1.5 mg QW Compound 1 for 4 weeks;
3 mg QW Compound 1 for 4 weeks;
6 mg QW Compound 1 for 4 weeks;
8 mg QW Compound 1 for 4 weeks; and
10 mg QW Compound 1 for 32 weeks.

The third dosing regimen (Cohort 3) is as follows:
1.5 mg QW Compound 1 for 4 weeks;
3 mg QW Compound 1 for 4 weeks;
6 mg QW Compound 1 for 4 weeks;
9 mg QW Compound 1 for 4 weeks;
12 mg QW Compound 1 for 4 weeks; and
16 mg QW Compound 1 for 28 weeks

Three cohorts are administered Compound 1, with one cohort administered 1.5 mg for Weeks 1-4, 3.0 mg for Weeks 5-32 and 6.0 mg for Weeks 33-48, a second cohort administered 1.5 mg for Weeks 1-4, 3.0 mg for Weeks 5-8, 6.0 mg for Weeks 9-12, 8.0 mg for Weeks 13-16 and 10.0 mg for Weeks 17-48, a third cohort administered 1.5 mg for Weeks 1-4, 3.0 mg for Weeks 5-8, 6.0 mg for Weeks 9-12, 9.0 mg for Weeks 13-16, 12.0 mg for Weeks 17-20, and 16.0 mg for Weeks 21-48.

### Assessments

Approximately 40 individuals per cohort are assessed at baseline and at Week 48 for change from baseline for multiple measures of body composition by magnetic resonance imaging analyses, including changes in liver fat, abdominal subcutaneous adipose tissue, and visceral adipose tissue. Individuals are assessed for changes in NAFLD, which is characterized by ≥10% liver fat by magnetic resonance imaging.

Blood samples are periodically collected from Week 1 through Week 48 to determine plasma concentrations across increasing doses of the Compound 1 in individuals with obesity.

HbA1c, C-peptide, glucagon, serum glucose and insulin are measured at fasting for multiple time points across dose and from Week 1 through Week 48. Mean absolute and percent change in fasting glucose, insulin, C-peptide, and glucagon are calculated at Week 32 and Week 48 and compared to pre-dose Week 1 baseline for each Compound 1, and placebo cohort. Mean absolute and percent change in fasting glucose, insulin, C-peptide, and glucagon are calculated for each Compound 1 and cohort and compared to placebo at Week 32 and Week 48.

Parameters derived from fasting samples to assess insulin resistance and pancreatic β cell function include homeostatic model assessment (HOMA)-B, HOMA-IR. Mean parameters for HOMA indices related to glycemic control, pancreatic beta-cell function, and insulin sensitivity will be derived from fasting measures for each Compound 1 cohort and compared to placebo at Week 32 and Week 48.

Satiety VAS is measured at fasting for multiple time points across dose and from Week 1 through Week 48. Appetite sensations are assessed using a VAS for parameters of hunger, fullness, satiety and prospective food intake, and an overall appetite score is calculated.

Body weight and waist circumference are measured at frequent intervals across dose and from Week 1 through Week 48 and for an extended follow-up period after the conclusion of dosing. Mean absolute and percent change in body weight are calculated at Week 32 and Week 48 and compared to pre-dose Week 1 baseline for each Compound 1 and placebo cohort. Mean absolute and percent change in waist circumference are calculated at Week 32 and Week 48 and compared to pre-dose Week 1 baseline for each Compound land Placebo cohorts. Mean absolute and percent change in waist circumference are calculated for each Compound 1 cohort and compared to Placebo at Week 32 and Week 48.

Lipid profiles, including total cholesterol, LDL cholesterol, VLDL cholesterol, HDL cholesterol, and triglycerides are measured at fasting for multiple time points across dose and from Week 1 through Week 48. Mean absolute and percent change in fasting lipid profiles, including total cholesterol, LDL cholesterol, VLDL cholesterol, HDL cholesterol, and triglycerides are calculated at Week 32 and Week 52 and compared to pre-dose Week 1 baseline for each Compound 1 and placebo cohort. Mean absolute and percent change in fasting lipid profiles, including total cholesterol, LDL cholesterol, VLDL cholesterol, HDL cholesterol, and triglycerides are calculated for each Compound 1 cohort and compared to placebo at Week 32 and Week 48.

Vital signs are monitored at frequent intervals across dose and from Week 1 through Week 48, including heart rate, pulse rate, systolic blood pressure, and diastolic blood pressure.

Safety parameters assessed during the study include: AEs (with special interest to GI AEs such as nausea, vomiting, and diarrhea), laboratory parameters, physical examinations/medical assessments, vital signs and ECGs, BW, injection site reactions, hypersensitivity reactions, and glucose monitoring (with special attention to mean parameters for HOMA indices related to glycemic control, pancreatic beta-cell function, and insulin sensitivity will be derived from fasting measures at Week 32 and Week 48 and compared to pre-dose Week 1 baseline for each Compound land placebo cohort.

1. A method for chronic weight management, improving glycemic control, and/or reducing LDL cholesterol or triglycerides in a patient in need thereof;
wherein said method comprises:
(1) administering a once weekly dose of a compound of SEQ ID NO:1, or a pharmaceutically acceptable salt thereof, to said patient for at least about two weeks; and
(2) thereafter increasing the dose to greater than about 6 mg to about 16 mg and administering the increased dose to said patient once weekly for at least about two weeks.

2. The method according to claim 1, wherein the once weekly increased dose is selected from the group consisting of about 6.25 mg to about 16.0 mg, from about 6.5 mg to about 16.0 mg of the compound, from about 8.0 mg to about 16.0 mg, from about 10 mg to about 16.0 mg, from about 11.0 mg to about 16 mg, from about 12.0 mg to about 16.0 mg, from about 13.0 mg to about 16.0 mg or from about 14.0 mg to about 16.0 mg.

3. The method according to claim 1, wherein the once weekly increased dose is about 6.25 mg, about 6.5 mg, about 7.0 mg, about 8.0 mg, about 9.0 mg, about 10.0 mg, about 11.0 mg, about 12.0 mg, about 13.0 mg, about 14.0 mg, about 15.0 mg or about 16.0 mg.

4. The method according to claim 2, wherein the once weekly increased dose is from about 8 mg to about 16.0 mg.

5. The method according to claim 4, wherein the once weekly increased dose is about 8.0 mg, about 9.0 mg, about 10.0 mg, about 11.0 mg, about 12.0 mg, about 13.0 mg, about 14.0 mg, about 15.0 mg or about 16.0 mg.

6. The method according to claim 2, wherein the once weekly increased dose is from about 10 mg to about 16.0 mg.

7. The method according to claim 6, wherein the once weekly increased dose is about 10.0 mg, about 11.0 mg, about 12.0 mg, about 13.0 mg, about 14.0 mg, about 15.0 mg or about 16.0 mg.

8. The method according to claim 1, wherein multiple different once weekly doses are administered to the patient in steps (1) and (2).

9. The method according to claim 8, wherein a first once weekly dose is about 1.0 mg to about 6.0 mg.

10. The method according to claim 9, wherein the first once weekly dose is about 1.0 mg, about 1.5 mg, about 2.0 mg, about 2.5 mg, about 3.0 mg, about 3.5 mg, about 4.0 mg, about 4.5 mg, about 5.0 mg, about 5.5 mg or about 6.0 mg.

11. The method according to claim 10, wherein the first once weekly dose is about 1.5 mg, about 2.0 mg or about 3.0 mg.

12. The method according to claim 9, wherein the first once weekly dose and each subsequent increased once weekly dose are increased in increments of about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg or any combination thereof, and wherein each dose is administered for at least about two weeks.

13. The method according to claim 12, wherein the first once weekly dose is about 1.5 mg, about 2.0 mg or about 3.0 mg and wherein the first once weekly dose and each subsequent increased once weekly dose are increased in increments of about 1.5 mg, about 2.0 mg, about 3.0 mg, about 4.0 mg or any combination thereof.

14. The method according to claim 1, comprising:
(a) administering a first dose of about 1.5 mg of the compound, or a pharmaceutically acceptable salt thereof, once weekly for at least about two weeks;
(b) increasing the dose to about 3.0 mg of the compound, or a pharmaceutically acceptable salt thereof, and administering the dose once weekly for at least about two weeks;
(c) increasing the dose to about 6.0 mg of the compound, or a pharmaceutically acceptable salt thereof, and administering the dose once weekly for at least about two weeks;
(d) increasing the dose to about 8.0 mg of the compound, or a pharmaceutically acceptable salt thereof, and administering the dose once weekly for at least about two weeks; and
(e) increasing the dose to about 10.0 mg of the compound, or a pharmaceutically acceptable salt thereof, and administering the dose once weekly for at least about two weeks.

15. The method according to claim 1, comprising:
(a) administering a first dose of about 2.0 mg of the compound, or a pharmaceutically acceptable salt thereof, once weekly for at least about two weeks;
(b) increasing the dose to about 4.0 mg of the compound, or a pharmaceutically acceptable salt thereof, and administering the dose once weekly for at least about two weeks;
(c) increasing the dose to about 6.0 mg of the compound, or a pharmaceutically acceptable salt thereof, and administering the dose once weekly for at least about two weeks;
(d) increasing the dose to about 8.0 mg of the compound, or a pharmaceutically acceptable salt thereof, and administering the dose once weekly for at least about two weeks; and
(e) increasing the dose to about 10.0 mg of the compound, or a pharmaceutically acceptable salt thereof, and administering the dose once weekly for at least about two weeks.

16. The method according to claim 1, comprising:
(a) administering a first dose of about 1.5 mg of the compound, or a pharmaceutically acceptable salt thereof, once weekly for at least about two weeks;
(b) increasing the dose to about 3.0 mg of the compound, or a pharmaceutically acceptable salt thereof, and administering the dose once weekly for at least about two weeks;
(c) increasing the dose to about 6.0 mg of the compound, or a pharmaceutically acceptable salt thereof, and administering the dose once weekly for at least about two weeks;
(d) increasing the dose to about 8.0 mg of the compound, or a pharmaceutically acceptable salt thereof, and administering the dose once weekly for at least about two weeks;
(e) increasing the dose to about 12.0 mg of the compound, or a pharmaceutically acceptable salt thereof, and administering the dose once weekly for at least about two weeks; and
(f) increasing the dose to about 16.0 mg of the compound, or a pharmaceutically acceptable salt thereof, and administering the dose once weekly for at least about two weeks.

17. The method according to claim 1, comprising:
(a) administering a first dose of about 2.0 mg of the compound, or a pharmaceutically acceptable salt thereof, once weekly for at least about two weeks;
(b) increasing the dose to about 4.0 mg of the compound, or a pharmaceutically acceptable salt thereof, and administering the dose once weekly for at least about two weeks;
(c) increasing the dose to about 6.0 mg of the compound, or a pharmaceutically acceptable salt thereof, and administering the dose once weekly for at least about two weeks;
(d) increasing the dose to about 8.0 mg of the compound, or a pharmaceutically acceptable salt thereof, and administering the dose once weekly for at least about two weeks;
(e) increasing the dose to about 10.0 mg of the compound, or a pharmaceutically acceptable salt thereof, and administering the dose once weekly for at least about two weeks;
(f) increasing the dose to about 13.0 mg of the compound, or a pharmaceutically acceptable salt thereof, and administering the dose once weekly for at least about two weeks; and
(g) increasing the dose to about 16.0 mg of the compound, or a pharmaceutically acceptable salt thereof, and administering the dose once weekly for at least about two weeks.

18. The method according to claim 1, comprising:
(a) administering a first dose of about 1.5 mg of the compound, or a pharmaceutically acceptable salt thereof, once weekly for at least about two weeks;
(b) increasing the dose to about 3.0 mg of the compound, or a pharmaceutically acceptable salt thereof, and administering the dose once weekly for at least about two weeks;
(c) increasing the dose to about 6.0 mg of the compound, or a pharmaceutically acceptable salt thereof, and administering the dose once weekly for at least about two weeks;
(d) increasing the dose to about 9.0 mg of the compound, or a pharmaceutically acceptable salt thereof, and administering the dose once weekly for at least about two weeks;
(e) increasing the dose to about 12.0 mg of the compound, or a pharmaceutically acceptable salt thereof, and administering the dose once weekly for at least about two weeks; and
(f) increasing the dose to about 16.0 mg of the compound, or a pharmaceutically acceptable salt thereof, and administering the dose once weekly for at least about two weeks.

19. The method according to claim 1, comprising:
(a) administering a first dose of about 1.5 mg of the compound, or a pharmaceutically acceptable salt thereof, once weekly for at least about two weeks;
(b) increasing the dose to about 3.0 mg of the compound, or a pharmaceutically acceptable salt thereof, and administering the dose once weekly for at least about two weeks;
(c) increasing the dose to about 6.0 mg of the compound, or a pharmaceutically acceptable salt thereof, and administering the dose once weekly for at least about two weeks;
(d) increasing the dose to about 10.0 mg of the compound, or a pharmaceutically acceptable salt thereof, and administering the dose once weekly for at least about two weeks;
(e) increasing the dose to about 13.0 mg of the compound, or a pharmaceutically acceptable salt thereof, and administering the dose once weekly for at least about two weeks; and
(f) increasing the dose to about 16.0 mg of the compound, or a pharmaceutically acceptable salt thereof, and administering the dose once weekly for at least about two weeks.

20. The method according to claim 1, comprising administering each once weekly dose for at least about four weeks.

21. The method according to claim 1, wherein the compound, or a pharmaceutically acceptable salt thereof, is administered subcutaneously.

22. The method according to claim 1, wherein the administration of the compound of SEQ ID NO:1, or a pharmaceutically acceptable salt thereof, results in a decrease in the patient's body weight.

23. The method according to claim 1, wherein the administration of the compound of SEQ ID NO:1, or a pharmaceutically acceptable salt thereof, results in a decrease in the patient's HbA1c.

24. The method according to claim 1, wherein the administration of the compound of SEQ ID NO:1, or a pharmaceutically acceptable salt thereof, results in a decrease in the patient's LDL cholesterol and/or triglycerides decreases.

25. The method according to claim 1, wherein the administration of the compound of SEQ ID NO:1, or a pharmaceutically acceptable salt thereof, does not result in unacceptable tolerability.

26. The method according to claim 1, wherein the patient is overweight.

27. The method according to claim 1, wherein the patient is obese.

28. The method according to claim 1, wherein the patient has type 2 diabetes.

29. A compound of SEQ ID NO:1, or a pharmaceutically acceptable salt thereof, for use in providing chronic weight management, improving glycemic control and/or reducing LDL cholesterol or triglycerides, wherein:
(1) a once weekly dose of the compound of SEQ ID NO:1, or a pharmaceutically acceptable salt thereof, is administered for at least about two weeks; and
(2) thereafter the once weekly dose in increased to greater than about 6 mg to about 16 mg and is administered for at least about two weeks.

30. (canceled)
